(19) United States
(12) Patent Application Publication
(10) Pub. No.: US 2023/0143051 A1
(43) Pub. Date: May 11, 2023

(54) REAL-TIME IR FUNDUS IMAGE TRACKING IN THE PRESENCE OF ARTIFACTS USING A REFERENCE LANDMARK

(71) Applicants: Carl Zeiss Meditec, Inc., Dublin, CA (US); Carl Zeiss Meditec AG, Jena (DE)

(72) Inventor: Homayoun BAGHERINIA, Oakland, CA (US)

(21) Appl. No.: 17/915,442

(22) PCT Filed: Apr. 29, 2021
(86) PCT No.: PCT/EP2021/061233

§ 371 Date: Sep. 28, 2022

Related U.S. Application Data
(60) Provisional application No. 63/017,576, filed on Apr. 29, 2020.

(51) Int. Cl. A61B 3/113; A61B 3/10; A61B 3/12; A61B 3/00; G06T 7/246

(52) U.S. Cl. CPC A61B 3/113; A61B 3/102; A61B 3/1225; A61B 3/0025; G06T 7/248; G06T 2207/30041; G06T 2207/10048; G06T 2207/20084; G06T 2207/30101; G06T 2207/30096; G06T 2207/10101

(57) ABSTRACT

A system and method for ophthalmic motion tracking. An anchor point and multiple auxiliary points are selected from a reference image. Individual live images in a series of images are then searched for matches of the anchor point and auxiliary point. First, the anchor point is found, and then searches for individual auxiliary points is limited to a search window defined by the known distance and/or orientation of the sought auxiliary point relative to the anchor point.

### FIELD OF INVENTION

The present invention is generally directed to motion tracking. More specifically, it is directed to ophthalmic motion tracking of the anterior segment and posterior segment of the eye.

### BACKGROUND

Fundus imaging, such as may be obtained by use of a fundus camera, generally provides a frontal planar view of the eye fundus as seen through the eye pupil. Fundus imaging may use light of different frequencies, such as white, red, blue, green, infrared (IR), etc. to image tissue, or may use frequencies selected to excite fluorescent molecules in certain tissues (e.g., autofluorescence) or to excite a fluorescent dye injected into a patient (e.g., fluorescein angiography). A more detailed discussion of different fundus imaging technologies is provided below.

OCT is a non-invasive imaging technique that uses light waves to produce cross-section images of retinal tissue. For example, OCT permits one to view the distinctive tissue layers of the retina. Generally, an OCT system is an interferometric imaging system that determines a scattering profile of a sample along an OCT beam by detecting the interference of light reflected from a sample and a reference beam creating a three-dimensional (3D) representation of the sample. Each scattering profile in the depth direction (e.g., z-axis or axial direction) may be reconstructed individually into an axial scan, or A-scan. Cross-sectional, two-dimensional (2D) images (B-scans), and by extension 3D volumes (C-scans or cube scans), may be built up from multiple A-scans acquired as the OCT beam is scanned/moved through a set of transverse (e.g., x-axis and y-axis) locations on the sample. OCT also permits construction of a planar, frontal view (e.g., en face) 2D image of a select portion of a tissue volume (e.g., a target tissue slab (sub-volume) or target tissue layer(s) of the retina). OCTA is an extension of OCT, and it may identify (e.g., renders in image format) the presence, or lack, of blood flow in a tissue layer. OCTA may identify blood flow by identifying differences over time (e.g., contrast differences) in multiple OCT images of the same retinal region, and designating differences that meet predefined criteria as blood flow. A more in-depth discussion of OCT and OCTA is provided below.

Real-time and efficient tracking of fundus images (e.g., infrared (IR) fundus images) is important in automated retinal OCT image acquisition. Retinal tracking is particularly crucial due to involuntary eye movements during image acquisition, and particularly between OCT and OCTA scans.

IR images can be used to track the motion of the retina. However, insufficient IR image quality and the presence of various artifacts can affect automated real-time processing and thereby reduce the success rate and reliability. The quality of IR images can vary significantly in time depending on fixation, focus, vignetting effect, eye lashes, stripes and central reflex artifacts. Therefore, there is a need for a method that can track the retina robustly using IR images in real time. FIG. 1 provides exemplary IR fundus images with various artifacts, including stripe artifacts 11, central reflex artifact 13, and eye lashes 15 (e.g., seen as dark shadows).

Current tracking systems use a reference image with a set of extracted landmarks from the image. Then, the tracking algorithm tracks the live images using the landmarks extracted from the reference image by searching for them in each live image. The landmark matching between the reference image and the live image are determined independently. Therefore, matching becomes a difficult problem due to the presence of artifacts in images such as stripe and central reflex artifacts. Typically, sophisticated image processing algorithms are required to enhance the images prior to landmark detection. The real-time performance of the tracking algorithm would suffer using these additional algorithms specifically if the tracking algorithm is required to perform on high resolution images for more accurate tracking.

In summary, prior art tracking systems use a reference fundus image with a set of extracted landmarks from the reference image. Then, the tracking algorithm tracks a series of live images using the landmarks extracted from the reference image by independently searching for each landmark in each live image. Landmark matches between the reference image and the live image are determined independently. Therefore, matching landmarks becomes a difficult problem due to the presence of artifacts (such as stripe and central reflex artifacts) in images. Sophisticated image processing algorithms are required to enhance the IR images prior to landmark detection. The addition of these sophisticated algorithms can hinder their use in real-time application, particularly if the tracking algorithm is required to perform on high resolution (e.g., large) images for more accurate tracking.

It is an object of the present invention to provide a more efficient system/method for ophthalmic motion tracking.

It is another object of the present invention to provide ophthalmic motion tracking in real time using high resolution images.

### SUMMARY OF INVENTION

The above objects are met in a method/system for eye tracking. Unlike prior art tracking systems, the present system does not search for matching landmarks independently. Rather, the present invention identifies a reference (anchor) point/template (e.g., landmark), and matches additional landmarks relative to the position of the reference point. Landmarks may then be detected in live IR images relative to the reference (anchor) point, or template, taken from a reference image.

The reference point may be selected to be a prominent anatomical/physical feature (e.g. optic nerve head (ONH), a lesion, or a specific blood vessel pattern, if imaging the posterior segment of the eye) that can be easily identified and expected to be present in subsequent live images. Alternatively, e.g., if no prominent and consistent anatomical feature is present (such as if imaging the anterior segment of the eye), the reference anchor point may be selected from a pool/group of candidate reference point based on the current state of a series of images. As the quality of the series of live images changes, or a different prominent feature comes into view, the reference anchor point is revised/changed accordingly. Thus, in this alternate embodiment, the reference anchor point may change over time dependent upon the images being captured/collected.

It is to be understood that the reference point, or template, may include one or more characteristic features (pixel identifiers) that together define (e.g., identify) the specific landmark (e.g. ONH, a lesion, or a specific blood vessel pattern) used as the reference physical landmark. The distance between the reference point and a selected landmark in the reference IR image and a live IR image remains constant (or their relative distances remain constant) in both images. Thus, the landmark detection in live IR image become a simpler problem by searching a small region (e.g., bound region or window of predefined/fixed size) a prescribed/specific distanced from the reference point. The robustness of landmark detection is improved/facilitated due to the constant distance between the reference point and the landmark position. As initial landmarks are matched, searches for additional landmarks may be further limited to specific directions/orientations/angles relative to the already matched landmarks (e.g., in addition to the specific distance).

No sophisticated image processing algorithms are required to enhance the IR images prior to the present landmark detection, thus enhancing the speed of the present method, particularly when dealing with high resolution images. That is, the real-time performance of the tracking algorithm on high resolution images for more accurate tracking is ensured.

In summary, the present invention may begin by detecting a prominent point (e.g. ONH location or other point such as a lesion) as a reference/anchor point in a selected reference IR image (or other imaging modality) using, for example, a deep learning or knowledge-based computer vision method. Additional templates/points offset from the reference point center are extracted from the reference IR image to increase a number of templates and corresponding landmarks in the IR image. Optionally, multiple reference/anchor points are possible for general purposes. For example, one may capture multiple images of the eye, including a reference image and one or more live image. Multiple reference-anchor points may then be defined in the reference image, along with defining one or more auxiliary points in the reference image. Then within a select live image, a plurality of initial matching points that match (all or part of) the plurality of reference-anchor points are identified, and the select live image is transformed to the reference image as a coarse registration based on (e.g., using) the identified plurality of reference-anchor points. After providing this coarse registration, one may search for a match of a select auxiliary point within a region based on (e.g., bounded within a search region/FOV/window based) the location of the select auxiliary point relative to the plurality of matched reference-anchor points. One can then correct for a tracking error between the reference image and the select live image based on their matched points. This approach may be helpful when there is a significant geometrical transformation between the reference and live image during tracking and for more complicated tracking systems. For instance, if there is a large rotation (or affine/projective relationship) between two images, then two or more anchor points can coarsely register two images first for more accurate search for additional landmarks. The tracking algorithm then tracks live IR images (or corresponding other imaging modality) using a template centered at the reference point and additional templates extracted from the reference IR image. Given that a set of templates is extracted from the reference IR image, their corresponding locations (as a set of landmarks) in the live IR images can be determined by template matching in a small region distanced from the reference point in the live IR image. All or a subset of matches can be used to compute a transformation (x and y shifts, rotation, affine, projective, and/or nonlinear transformation) between the IR reference image and the live IR image. In this manner, landmarks (e.g., matching templates or matching points) are detected relative to a reference landmark. This leads to a real-time operation (e.g., processing is limited to a small region of an image) and robust tracking (e.g., false positives are eliminated as the distance between the reference landmark and a landmark is known, and provides an additional check for verifying the validate of a candidate match).

An advantage of the present method, as compared to the prior art, is that the landmarks are searched for, and detected, in specific areas/windows in a live image whose position and/or size is defined relative to the reference landmark (landmarks are tracked dependently). Thus, real-time tracking with minimal preprocessing on the high-resolution IR images can be implemented, and no sophisticated image processing techniques are required during tracking. The present invention is also less sensitive to the presence of image artifacts (such as stripes artifacts, central reflex artifact, eye lashes, etc.) due to tracking being defined relative to the reference point.

The present invention may further be extended to move a fundus image (e.g., an IR image) tracking region/window with a given FOV that at least partly overlaps an OCT's scan FOV. The IR FOV may be moved about (while maintaining an overlap with the OCT FOV) until achieving position that includes largest number of (or a sufficient number of) easily/robustly identifiable anatomical features (e.g. ONH, a lesion, or a specific blood vessel pattern) for maintaining robust tracking. This tracking information may then be used to provide motion compensation to the OCT system for OCT scanning.

Thus, the reference image may be used to align and trigger an auto-capture (e.g., from an OCT system) when the eye is stable (with no motion or minimal motion), and a sequence of retinal images are tracked robustly.

The present invention provides various metrics to quantifying the quality of tracking images, and possible causes of bad tracking images. Therefore, the present invention may extract from historical data various statistics for identifying various characteristic issues that affect tracking. For example, the present invention may analyze images sequences used for tracking and determining if the images are characteristic of systemic movement, random movement, or good fixation. An ophthalmic system using the present invention may then inform the system operator or a patient of the possible issue affecting tracking, and provide suggested solutions.

Other objects and attainments together with a fuller understanding of the invention will become apparent and appreciated by referring to the following description and claims taken in conjunction with the accompanying drawings.

Several publications may be cited or referred to herein to facilitate the understanding of the present invention. All publications cited or referred to herein, are hereby incorporated herein in their entirety by reference.

The embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

In the drawings wherein like reference symbols/characters refer to like parts:

FIG. 1 provides exemplary infrared (IR) fundus images with various artifacts, including stripe artifacts 11, central reflex artifact 13, and eye lashes 15 (e.g., seen as dark shadows).

FIGS. 2 and 3, show examples of tracking frames (each including an exemplary reference image and an exemplary live image) wherein a reference point and a set of landmarks (from the reference image) are tracked in a live IR image.

FIG. 4 illustrates the present tracking system in the presence of eye lashes 31 and central reflex 33 in the live IR image 23.

FIG. 5 shows two additional examples of the present invention.

FIG. 6 shows test resultant statistics for the registration error and eye motion for different acquisition modes and motion level.

FIG. 7 provides exemplary, anterior segment images with changes (in time) in pupil size, iris patterns, eyelid and eyelashes motion, and lack of contrast in eyelid areas within the same acquisition.

FIGS. 8 and 9 illustrate the tracking of the reference point and a set of landmarks in live images.

FIG. 10 illustrates an example of a tracking algorithm in accord with an embodiment of the present invention.

FIG. 11 provides tracking test results for the embodiment of FIG. 10.

FIG. 12 illustrates the use of ONH to determine the position of an optimal tracking FOV (tracking window) for a given OCT FOV (acquisition/scan window).

FIG. 13 illustrates a second implementation of the present invention for determining an optimal tracking FOV position without using the OHN or other predefined physiological landmark.

FIGS. 14A, 14B, 14C, and 14D provide additional examples the present method for identifying an optimal tracking FOV relative to the OCT FOV.

FIG. 15 illustrates scenario-1, wherein a reference image of the retina from previous visits for a patient fixation is available.

FIG. 16 illustrates scenario-2, where a retinal image quality algorithm detects the reference image during initial alignment (by the operator or automatic).

FIG. 17 illustrates scenario-3, where the reference image from previous visits and the retinal image quality algorithm are not available.

FIG. 18 illustrates an alternative solution for scenario-3.

FIG. 19A shows two examples for small (top) and normal (bottom) pupil acquisition modes.

FIG. 19B shows statistics for the registration error, eye motion and number of keypoints for a total number of 29,529 images from 45 sequences of images.

FIG. 20A illustrates the motion of a current image (white boarder) relative to a reference image (gray boarder) with eye motion parameters of Δx, Δy, and rotation φ relative to the reference image.

FIG. 20B shows an example from three different patients, one good fixation, another with systematic eye movement, and the third with random eye movement.

FIG. 21 provides a table that shows the statistics of eye motion for 15 patients.

FIG. 22 illustrates an example of a slit scanning ophthalmic system for imaging a fundus.

FIG. 23 illustrates a generalized frequency domain optical coherence tomography system used to collect 3D image data of the eye suitable for use with the present invention.

FIG. 24 shows an exemplary OCT B-scan image of a normal retina of a human eye, and illustratively identifies various canonical retinal layers and boundaries.

FIG. 25 shows an example of an en face vasculature image.

FIG. 26 shows an exemplary B-scan of a vasculature (OCTA) image.

FIG. 27 illustrates an example of a multilayer perceptron (MLP) neural network.

FIG. 28 shows a simplified neural network consisting of an input layer, a hidden layer, and an output layer.

FIG. 29 illustrates an example convolutional neural network architecture.

FIG. 30 illustrates an example U-Net architecture.

FIG. 31 illustrates an example computer system (or computing device or computer).

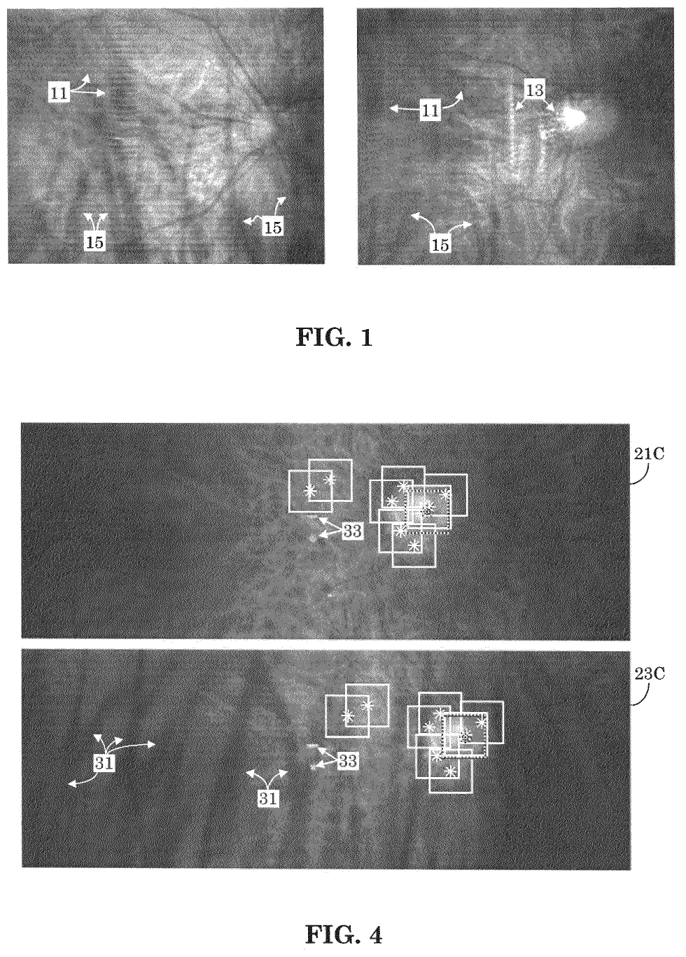

### DESCRIPTION OF THE PREFERRED EMBODIMENTS

The present invention provides an improved eye tracking system, such as for use in fundus cameras, optical coherence tomography (OCT) systems, and OCT angiography systems. The present invention is herein described using an infrared (IR) camera that tracks any eye in a series of live images, but it is to be understood that the present invention may be implemented using other imaging modalities (e.g., color images, fluorescent images, OCT scans, etc.). The present tracking system/method may begin by first identifying/detecting a (e.g., prominent) reference point (e.g., prominent physical feature that can be consistently (e.g., reliably and/or easily and/or quickly) identified in images. For example, the reference point (or reference template) may correspond to the optic nerve head (ONH) (and its reference location) or to another prominent/consistent point/feature, such as a lesion. The reference point may be selected from a reference IR image using a deep learning or other knowledge-based computer vision method.

Alternatively, or in addition, a series of candidate points may be identified in a stream of images, and the most consistently candidate point within a set of images may be selected as the reference anchor point for a series of live images. In this manner, the anchor point/template used in a series of live images may change as the quality of the live image stream changes and a different candidate point becomes more prominent/consistent.

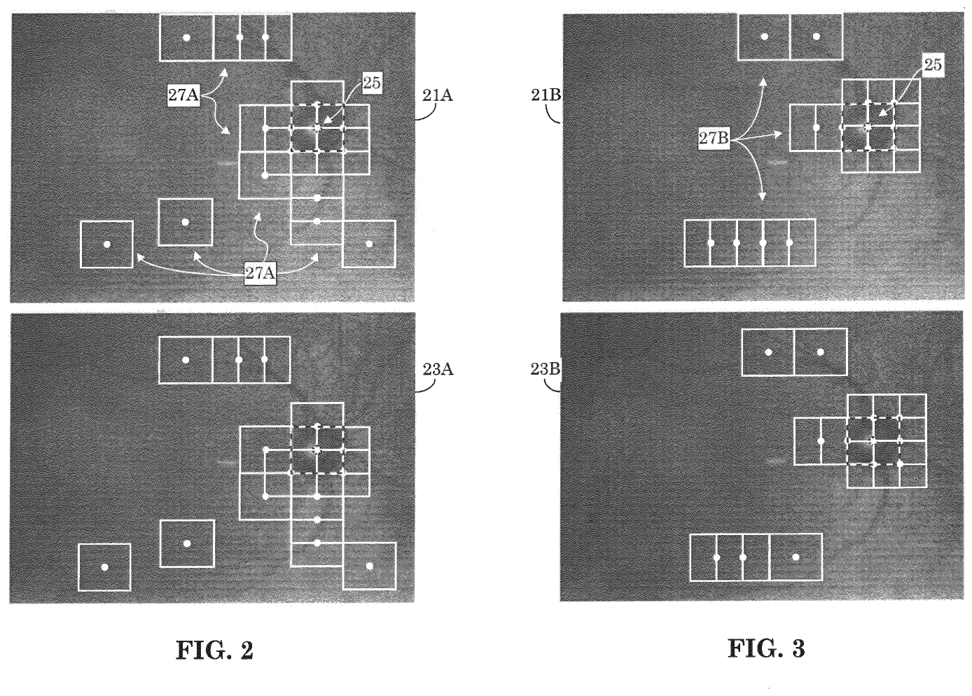

The present tracking algorithm tracks the live IR images using a template centered at the reference point extracted from the reference IR image. Additional templates offset from the reference point center are extracted to increase the number of templates and corresponding landmarks in the IR image. These templates can be used to detect the same positions in a different IR image as a set of landmarks which can be used for registration between the reference image and a live IR image which leads to tracking of a sequence of IR images in time. The advantage of generating a set of templates by offsetting the reference position is that no vessel enhancement or sophisticated image feature detection algorithms are required. The real-time performance of the tracking algorithm would suffer using these additional algorithms specifically if the tracking algorithm is required to perform on high resolution images for more accurate tracking. Given that a set of templates is extracted from the reference IR image, their corresponding locations (as a set of landmarks) in the live IR images can be determined by template matching (e.g. normalized cross correlation) in a small bound region distanced from the reference point in the live IR image. Optionally, one may search more templates if a match is not made with the initial set of templates. Once the corresponding matches are found, all or a subset of matches can be used to compute the transformation (x and y shifts and rotation) between the IR reference image and the live IR image. Optionally, if the number of matches is not greater than a threshold (e.g., half of the identified landmarks), the current live image is discarded and not corrected for tracking error. Assuming that sufficient matches are found, the transformation determines the amount of motion between the live IR image and the reference image. Theoretically, the transformation can be computed with two corresponding landmarks (the reference point and a landmark with high confidence) in the IR reference and a live image. However, more than two landmarks will be used for tracking to ensure a more robust tracking. FIGS. 2, 3, and 4 show examples of tracking frames (each including an exemplary reference image and an exemplary live image) wherein a reference point and a set of landmarks (from the exemplary reference image) are tracked in a live IR image. In each of FIGS. 2, 3, and 4, the top image (21A, 21B, and 21C, respectively) in each tracking frame is the exemplary reference IR image and the bottom image (23A, 23B, and 23C, respectively) is the exemplary live IR image. The dotted box is the ONH template, white boxes are the corresponding templates in both IR reference images and live images. The templates may be adaptively selected for each live IR image. For example, FIGS. 2 and 3 show the same reference image 21A/21B and the same anchor point 25, but the additional landmarks 27A in FIG. 2 are different than the additional landmarks 27B in FIG. 3. In this case, the landmarks are selected in each IR live image dynamically based on their detection confidence.

FIG. 4 illustrates the present tracking system in the presence of eye lashes 31 and central reflex 33 in the live IR image 23. Note that tracking is does not rely on presence of blood vessels in this case (as opposed to the examples of FIGS. 2 and 3), and thus avoids confusing eye lashes for blood vessels.

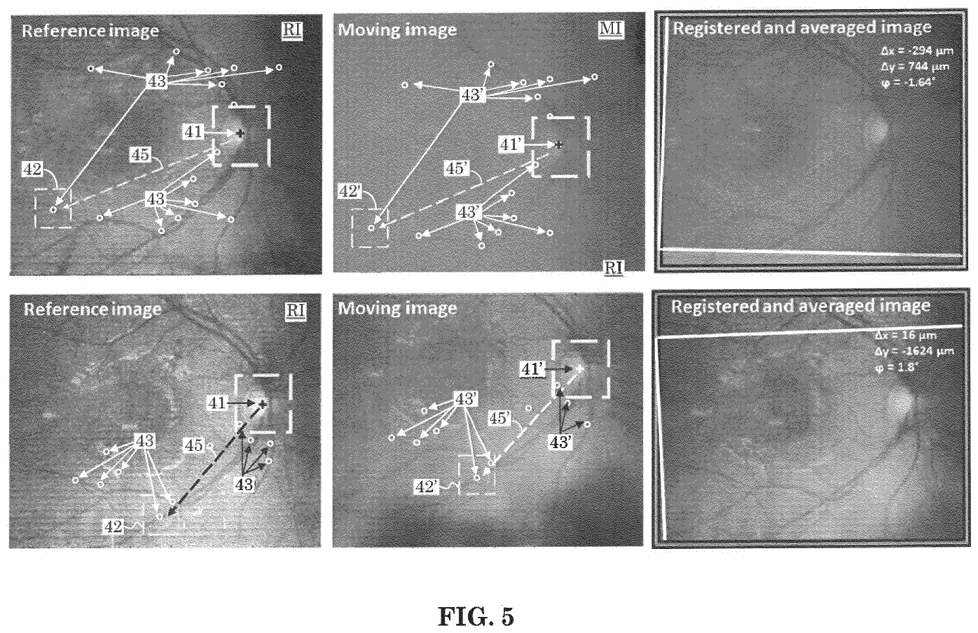

FIG. 5 shows two additional examples of the present invention. The top row of images show the present invention applied to normal pupil acquisition, and the bottom row of images show the present invention applied to small pupil acquisition. In both cases, landmarks are detected relative to the ONH location (e.g., reference-anchor point). In the example, tracking parameters (xy-translation and rotation) are calculated by registration between a reference image (RI) and a moving image (MI). The present method requires the ONH 41 location and a set of RI landmarks (e.g., auxiliary points) 43 extracted from feature rich areas of the reference image RI. For exemplary purposes, one of the landmarks 43 is shown within a bound region, or search window, 42 and its relative distance 45 to the ONH 41. The ONH 41 in reference image RI may be detected using a neural networks system having a U-Net architecture. A general discussion of neural networks, including a U-Net architecture, is provided below. The ONH 41′ in a moving (e.g., live) image MI may be detected by template-matching using the ONH template 41 extracted from the reference image RI. Each reference landmark template 43 and its relative distance 45 (and optionally its relative orientation) to the ONH 41 are used to search for corresponding landmarks 43′ (e.g., within a bound region, or window, 42′) having the same/similar distance 45′ from the ONH 41′ in a moving image MI. A subset of landmark correspondences with high confidence is used to compute the tracking parameters.

In an exemplary implementation, infrared (IR) images (11.52×9.36 mm with a pixel size of 15 μm/pixel) using a CLARUS 500 (ZEISS, Dublin, Calif.) at 50 Hz frame rate were collected, using normal and small pupil acquisition modes with induced eye motion. Each eye was scanned using 3 different motion levels, as follows: good fixation, systematic, and random eye movement. The registered images were displayed in a single image to visualize the registration (see FIG. 5). The mean distance error between the registered moving and reference landmarks was calculated as the registration error. The statistics of registration error and eye motion were reported for each acquisition mode and each motion level. Around 500 images were collected from 15 eyes.

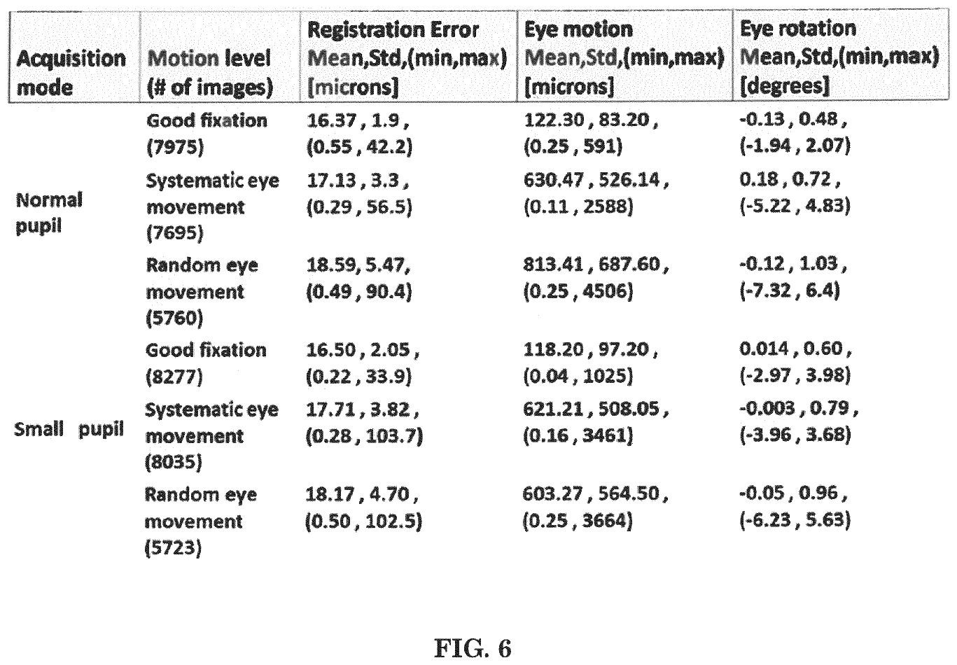

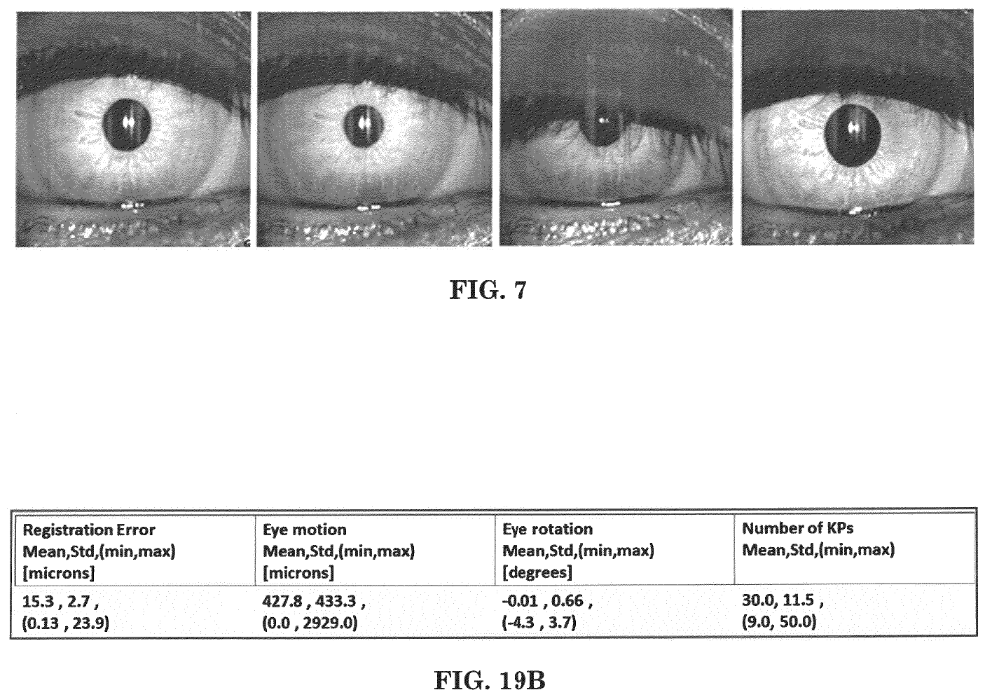

FIG. 6 shows the resultant statistics for the registration error and eye motion for different acquisition modes and motion level using all eyes. The mean and standard deviation of registration error for normal and small pupil acquisition modes are similar which indicates that the tracking algorithm has a similar performance for both modes. Reported registration errors are important information which help to design an OCT scan pattern. The tracking time for a single image was measured 13 ms on average using a computing system with an Intel i7 CPU, 2.6 GHz, and 32 GB RAM. Thus, the present invention provides for a real-time retinal tracking method using IR images with a demonstrated good tracking performance, which is an important part of an OCT image acquisition system.

Although the above example is described as applied to the posterior of the eye (e.g., fundus), it is be understood that the present invention may also be applied to the other parts of the eye, such as the anterior segment of the eye. Real-time and efficient tracking of anterior segment images is important in automated OCT angiography image acquisition. Anterior segment tracking is crucial due to involuntary eye movements during image acquisition, and particularly in OCTA scans. Anterior segment LSO images can be used to track the motion of the anterior segment of the eye. The eye motion may be assumed to be a rigid motion with motion parameters such as translation and rotation that can be used to steer an OCT beam. The local motions and lack of contrast of the front of the eye (such as pupil size/shape changes, consistent eye lashes and eyelids motion, squeezed or expanded iris patterns (due to pupil size/shape changes) during tracking) can affect automated real-time processing and thereby reduce the success rate and reliability of the tracking. Additionally, the appearance of anatomical features in images can vary significantly over time depending on a subject's fixation (e.g., gaze angle). FIG. 7 provides exemplary, anterior segment images with changes (in time) in pupil size, iris patterns, eyelid and eyelashes motion, and lack of contrast in eyelid areas within the same acquisition. Thus, there is a need for a method that can track the anterior portion of the eye robustly using LSO or other imaging modalities in real time.

Previous anterior segment tracking systems use a reference image with a set of extracted landmarks from the image. Then, a tracking algorithm tracks a series of live images using the landmarks extracted from the reference image by independently searching for the landmarks in each live image. That is, matching landmarks, between the reference image and a live image, are determined independently. Independent matching of landmarks between two images, assuming a rigid (or affine) transformation, becomes a difficult problem due to local motion and lack of contrast. Sophisticated landmark matching algorithms have typically been required to compute the rigid transformation. The real-time tracking performance of such a previous approach typically suffers if high resolution images are needed for more accurate tracking.

The above-described tracking embodiment (e.g., see FIGS. 2 to 6) provide efficient landmark match detection between two images, but some implementations may have a limitation. Some of the above-described embodiment(s) assumes that the reference and live images contain an obvious or unique anatomical feature, such as the ONH, which is robustly detectable due to the uniqueness of the anatomical feature. In this approach, landmarks are detected relative to a reference (anchor) point (e.g. ONH, a lesion, or a specific blood vessel pattern) in live images. The distance (or relative distance) between the reference point and a selected landmark in the reference image and a live image remains constant in both images. Thus, landmark detection in live image becomes a simpler problem by searching a small region a known distanced (and optionally, orientation) from the reference (anchor) point. The robustness of landmark detection is ensured due to the constant distance between the reference point and the landmark position.

By contrast, the present embodiment provides a few advantages over the above-described embodiment(s). Like in the above-described embodiment(s), a reference (e.g., anchor) point is selected from candidate landmarks extracted from a reference image, but the selected reference anchor point may not necessarily be an obvious or unique anatomical/physical feature (e.g. ONH, pupil, iris boundary or center) of the eye. Although the anchor point might not be a unique anatomical/physical feature, the distance between the reference anchor point and a selected auxiliary landmark in the reference image and a live image remains constant in both images. Thus, landmark detection in the live image become a simpler problem by searching a small region a known distance from the reference anchor point. The robustness of landmark detection is ensured due to the constant distance between the reference anchor point and the landmark auxiliary point. A subset of best matching landmarks may be selected by an exhaustive search of a subset to compute a rigid transformation. A similar approach may also be applied to retinal tracking using IR images (the above-described embodiment(s)) where the unique anatomical landmark is not visible (or not found) in an image/scan area or within a field of view (e.g. periphery) of a detector.

A difference of the present embodiment, as compared to some of the above-described embodiments, is that the reference (anchor) point is selected from a group of landmarks candidates extracted from a reference image. The reference point may be selected/chosen based on, for example, being trackable in following images (e.g., in a stream of images) to ensure the consistent and robust detection of this point. Basically, temporal image information (e.g., changes in a series of images over time) is incorporated into the reference point selection method. For example, all or a select image in a series of images (e.g., selected at fixed or variable intervals) may be examined to determine if the current landmark is still the best landmark for use as the reference anchor landmark. As a different landmark candidate becomes more trackable (e.g., more easily, quickly, uniquely, and/or consistently detectable), it replaces the previous reference point and becomes the new reference anchor point. All other landmark points may then be re-referenced relative to the new reference anchor point.

The present embodiment is particularly useful for situations where the scan (or image) area of the eye (the field of view) does not contain an obvious or unique anatomical feature (e.g., the ONH), or the anatomical feature is not necessarily useful to be selected as a reference point (e.g., pupil due to its size/shape changing during tracking, e.g., changing over time). Thus, in the present embodiment, uniqueness of the anatomical feature to be selected as a reference point is not required.

The present embodiment may first detect a reference (anchor) point from a set of candidate landmarks extracted from a reference image or a series of consecutive live images. For instance, the reference landmark candidates may be within a region having great texture properties, such as iris regions towards the outer edge of the iris. An entropy filter may highlight the regions with great texture properties followed by additional image processing and analysis techniques to generate a mask that contains the landmark candidates to be selected as a reference point candidate. The reference point located in an area with high contrast and texture, which is trackable in the following live images, can be selected as the reference (anchor) point. A deep learning (e.g., neural network) method/system may be used to identify image regions with high contrast and great texture properties.

The present embodiment tracks live images using a template centered at the reference (anchor) point extracted from the reference image. Additional templates centered at the landmark candidates, which are extracted from the reference image, are generated. These templates can be used to detect the same positions in a live image as a set of landmarks which can be used for registration between the reference image and a live image which leads to tracking of a sequence of images over time. Given that a set of templates is extracted from the reference image, their corresponding locations (as a set of landmarks) in the live images can be determined by template matching (e.g. normalized cross correlation) in a small region distanced from the reference point in the live image. Once all the corresponding matches are found, a subset of matches can be used to compute the transformation (x and y shifts and rotation) between the reference image and the live image.

The transformation determines the amount of motion between the live image and the reference image. The transformation may be computed with two corresponding landmarks in the reference and a live image. However, more than two landmarks may be used for tracking to ensure the robustness of tracking.

The subset of matching landmarks may be determined by an exhaustive search. For example, at each iteration, two pairs of corresponding landmarks may be selected from the reference and live images. A rigid transformation may then be calculated using the two pairs. The error between each transformed reference image landmark (using rigid transform) to live image landmark is determined. The landmarks associated with an error smaller than a predefined threshold may be selected as inliers. This procedure may be repeated for all (or most) passible (e.g., combinations of) two pairs. The transformation that creates maximum number of inliers may then be selected as the rigid transformation for tracking.

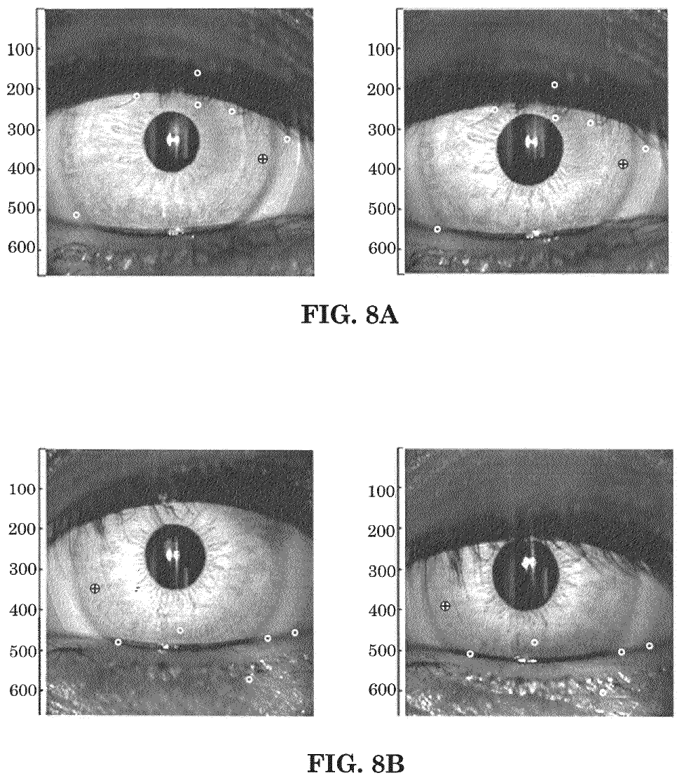

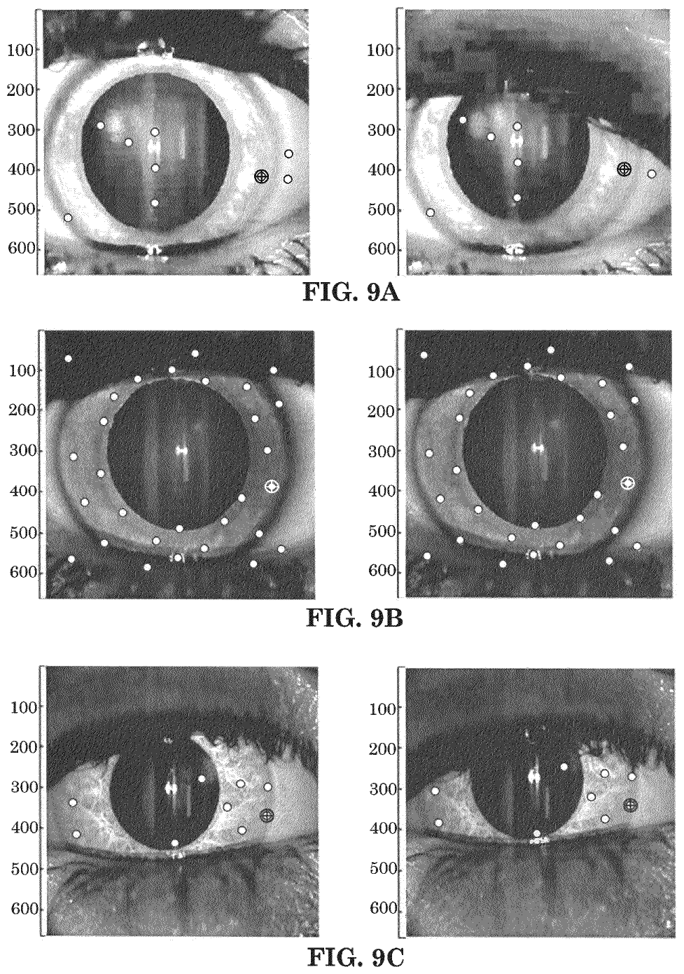

FIGS. 8 and 9 illustrate the tracking of the reference point and a set of landmarks in live images. The images in the left column are reference images and the images in the right column are live images. The selected reference point is marked with an encircled cross each reference image. As is seen, selection of the reference anchor point changes over time as different parts of a live image stream change (e.g., in shape or quality). Changes in pupil size and shape, eyelid motion, and low contrast are visible in the examples of FIGS. 8 and 9.

In summary, motion artifacts pose a challenge in optical coherence tomography angiography (OCTA). While motion tracking solutions that correct these artifacts in retinal OCTA exist, the problem of motion tracking is not yet solved for the anterior segment (AS) of the eye. This currently is an obstacle to the use of AS-OCTA for diagnosis of diseases of the cornea, iris and sclera. The present embodiment has been demonstrated for motion tracking of the anterior segment of an eye.

In a particular embodiment, a telecentric add-on lens assembly with internal fixation was used to enable imaging of the anterior segment with a CIRRUS™ 6000 AngioPlex (ZEISS, Dublin, Calif.) with good patient alignment and fixation (fx). Using this add-on lens on the CIRRUS 6000, widefield (20×14 mm) line scanning ophthalmoscope (LSO) image sets were taken of 25 eyes from 15 subjects—6973 images in total (4798 central & 2175 peripheral fixation). Motion in these image sets was then tracked with an algorithm using real-time landmark-based rigid registration between a reference image and the other (moving) images from the same set.

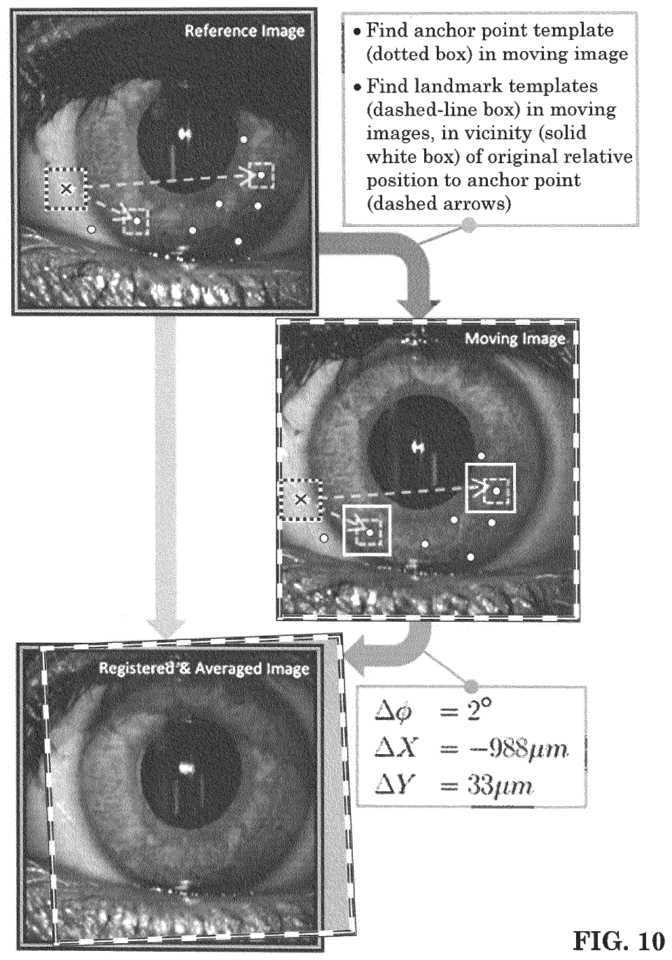

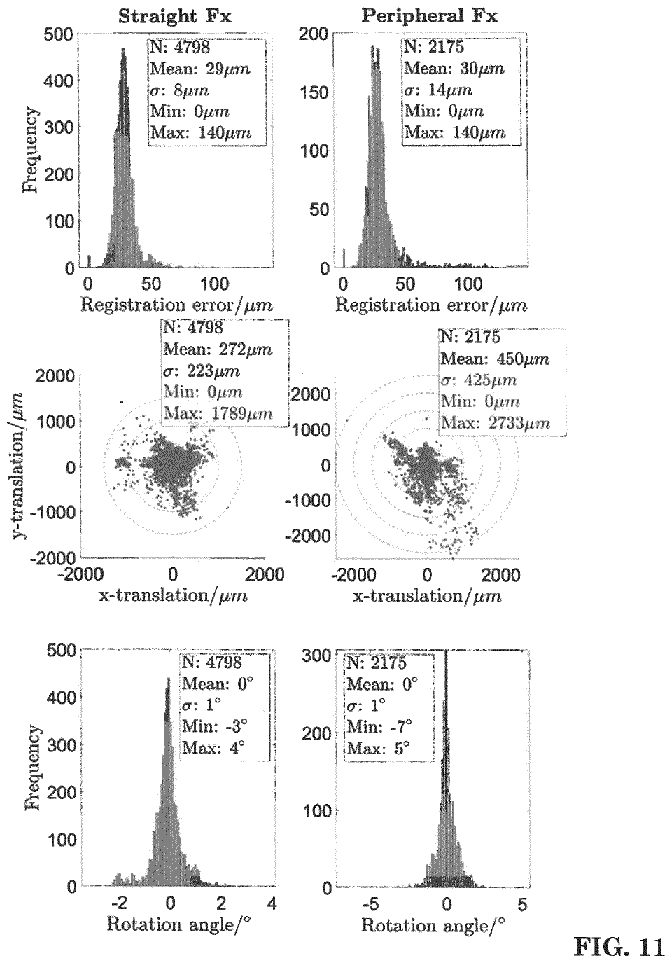

FIG. 10 illustrates an example of a tracking algorithm in accord with an embodiment of the present invention. In the present example, the anchor point and selected landmarks are found in the moving image and used to calculate translation and rotation values for registration. The overlade images at the bottom in FIG. 10 are shown for visual verification. The present embodiment first detects an anchor point in an area of the reference image with high texture values. This anchor point is then located in the moving image by searching for a template (image region) centered at the reference image anchor point position. Next, landmarks from the reference image are found in the moving image by searching for landmark templates at the same distance to the anchor point as in the reference image. Finally, translation and rotation are calculated using the landmark pairs with the highest confidence values. The registration error is the mean distance between corresponding landmarks in both images. This value is calculated after visual confirmation of landmark matches and successful registration. FIG. 11 provides tracking test results. The insets in the registration error and rotation angle histograms show the respective distribution parameters. The translation vectors are plotted in the center, with concentric rings every 500 μm of magnitude. The insets show the distribution parameters for the magnitude of translation.

As mentioned above, real-time and efficient tracking of IR fundus images is important in automated retinal OCT image acquisition. Retinal tracking becomes more challenging when the patient fixation is not straight or is off-centered, assuming the tracking and OCT acquisition field of view (FOV) are placed on the same retinal region. That is, the tracking and OCT (acquisition) FOV are usually located on the same area on the retina. In this way, the motion tracking information can be used, for example, to correct OCT positioning during an OCT scan operation. However, the location where the OCT scan is being conducted (e.g., the OCT acquisition FOV, or OCT FOV) may not be at a location on the retina with sufficient characteristic physical features/structures track motion in robust manner. The presently preferred approach identifies/determines an optimal tracking FOV position prior to tracking and OCT acquisition.

There are several challenges that complicate efficient tracking. For example, the IR images might not contain enough distributed retinal features (such as blood vessels, etc.) to track when the fixation is off-centered. Another complication is that eye curvature is larger in the periphery region of the eye. Furthermore, eye motion can create more nonlinear distortion in the current image relative to the reference image, which may lead to inaccurate tracking. The transformation between the current image relative to the reference image may not be a rigid transformation due to a nonlinear relationship between two images.

One possible solution would be to place the tracking FOV where there are well-defined retinal landmarks and features (e.g. around ONH and large blood vessels) that can be detected robustly. A problem with this approach is that a large distance between the tracking FOV and the OCT acquisition FOV introduces a rotation angle error due to the location of the rotation anchor point being in the tracking FOV and not in OCT acquisition FOV.

To overcome the above challenges, the tracking FOV (e.g., in the IR image) can be made to at least partially overlapped with the OCT acquisition FOV for off-centered fixations, or it can be placed as close as possible to the OCT acquisition FOV.

Herein is presented a method for optimal and dynamic positioning of the tracking FOV for a patient fixation. In the present approach, a tracking algorithm (such as described above, or other suitable tracking algorithm) is used to optimize the position of the tracking FOV by maximizing the tracking performance using a set of metrics, such as tracking error, landmark (keypoints) distribution as well as the number of landmarks. The tracking position (center of FOV) that maximizes the tracking performance is selected/designate/identified as the desired position for a given patient fixation.

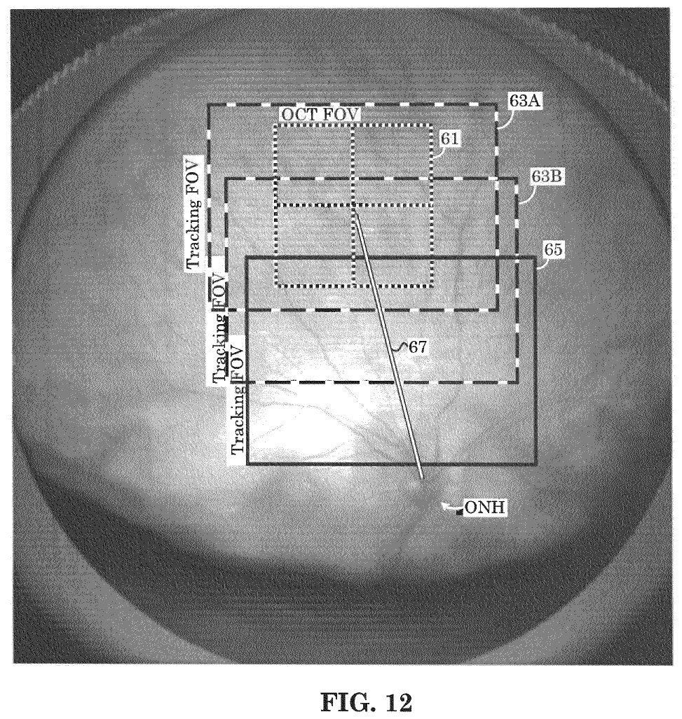

In essence, the present invention dynamically finds an optimal tracking FOV (for a given patient fixation) that enables good tracking for an OCT scan of an off-center fixation (e.g., fixation at a periphery region of the eye). In the present approach, the tracking area can be placed on a different location on the retina than the OCT scanning area. An optimal area on the retina relative to the OCT FOV is identified and used for retinal tracking. The tracking FOV can thus be found dynamically for each eye. For example, the optimal tracking FOV may be determined based on the tracking performance on a series of alignment images. FIG. 12 illustrates the use of ONH to determine the position of an optimal tracking FOV (tracking window) for a given OCT FOV (acquisition/scan window). In the present examples, the IR preview images (or a section/window within the IR preview images), which typically have a wide FOV (e.g., 90-degree FOV) are used for patient alignment, may also be used to define the tracking FOV. These images along with the IR tracking algorithm can be used to determine the optimal tracking location relative to the OCT FOV. Dotted-outline box 61 defines the OCT FOV, dashed-outline boxes 63A and 63B indicate a moving (repositioning) non-optimal tracking FOV that is moved until an optimal tracking FOV (solid black-outline box 65) is identified. The non-optimal tracking FOV 63A/63B moves towards the ONH within a distance to the OCT FOV center (e.g., indicated by solid white line 67). The optimal tracking FOV (solid black-outline box 65) in the reference IR preview image enables a robust tracking of the remaining IR preview images in the same tracking FOV.

Two embodiments (or implementations) of the present invention are provided herein. The first embodiment uses a reference point. This implementation relies on the reference point on the retina that is detectable. The reference point, for example, can be the center of the optic nerve head (ONH). The implementation may be summarized as follows:

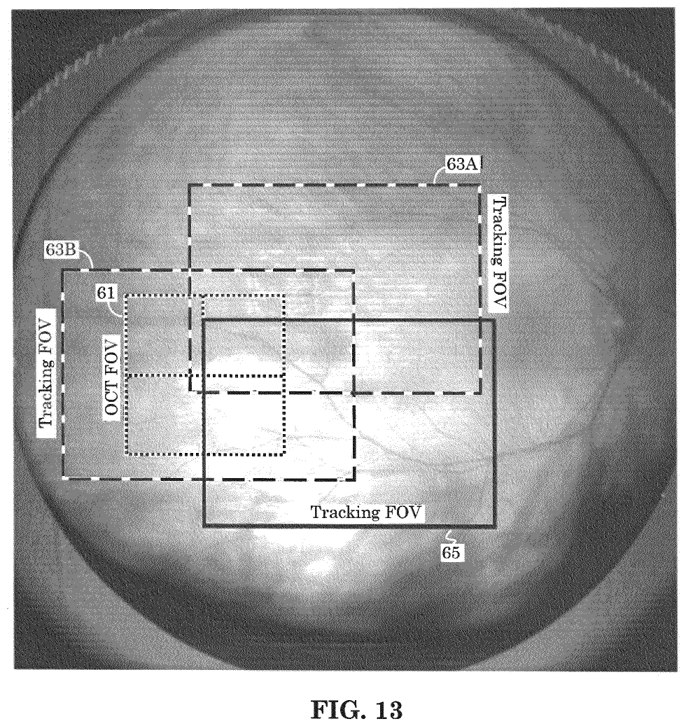

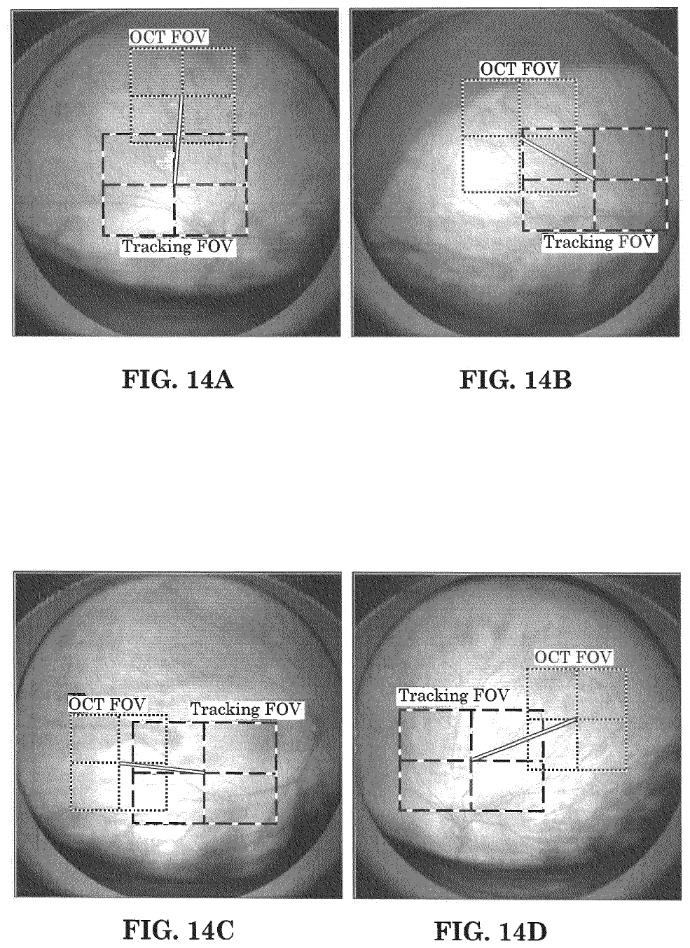

1) Collect a series of wide (e.g., 90-degree) FOV IR preview images (such as used in patient alignment), or other suitable fundus images.
2) Use/designate one of the collected images as the reference image.
3) Detect the ONH center in the reference image.
4) Crop the tracking FOV at the center of the OCT FOV in the reference image and use the cropped FOV as the tracking reference image (e.g., the current non-optimal tracking FOV 63A/63B).
5) Track remaining IR preview images in the set using the tracking reference image.
6) Update an objective function. As it is known in the art, the objective function in a mathematical optimization problem is the real-valued function whose value is to be either minimized or maximized over the set of feasible alternatives. In the present case, the objective function value is updated using tracking outputs such as tracking error, landmark distribution, number of landmarks, etc. of all remaining IR preview images.
7) Update the tracking reference image by cropping the tracking FOV along a connecting line (between tracking and OCT FOV centers) towards the ONH center, e.g., line 67. An alternative to the connecting line can be a nonlinear dynamic path from the tracking FOV center to the OCT FOV center. The nonlinear dynamic path can be determined for each scan/eye.
8) Repeat step 5) to 8) until the objective function is minimized for a maximum allowed distance between the OCT FOV and optimal tracking FOV (e.g., constrained optimization). FIG. 13 illustrates a second implementation of the present invention for determining an optimal tracking FOV position without using the OHN or other predefined physiological landmark. All elements similar to those of FIG. 12 have similar reference characters and are described above. This approach searches for the optimal tracking FOV 65 around the OCT FOV 61. The optimal position (solid black-outline box) of the tracking FOV in the reference IR preview image enables a robust tracking of the remaining IR preview images in the same tracking FOV. FIGS. 14A, 14B, 14C, and 14D provide additional examples the present method for identifying an optimal tracking FOV relative to the OCT FOV.

The second proposed solution/embodiment uses no reference point. This approach can be summarized as follows:

1) Collect a series of wide FOV IR preview images (e.g., such as used in patient alignment).
2) Use one of the images as the reference image.
3) Crop the tracking FOV at the center of the OCT FOV in the reference image and use the cropped FOV as the tracking reference image.
4) Track remaining IR preview images in the set relative to the tracking reference image.
5) Update the objective function value using tracking outputs such as tracking error, landmark distribution, number of landmarks, etc. of all remaining IR preview images.
6) Update the tracking reference image by cropping the tracking FOV towards the areas of the IR preview reference image with rich anatomical features such as blood vessels and lesions. The image saliency approaches can be used to update the tracking FOV position.
7) Repeat step 4) to 7) until the objective function is minimized for a maximum allowed distance between OCT and tracking FOV (constrained optimization).

The above-described retinal tracking methods may also be used for auto-capture (e.g., of an OCT scan and/or fundus image). This would be in contrast to prior art methods that use pupil tracking for auto-alignment and capture, but no prior art approach is known the inventors of using retinal tracking for alignment and auto-capture.

Automated patient alignment and image capture creates a positive and effective operator and patient experience. After initial alignment by an operator, the system can engage an automated tracking and OCT acquisition. Fundus images may be used for aligning the scan region on the retina. However, automated capture can be a challenge due to: eye motion during alignment; a blink and partial blink; and alignment stability that can cause the instrument to become misaligned quickly, such as due to eye motion, focus, operator error, etc.

A retinal tracking algorithm can be used to lock onto the fundus image and track the incoming moving images. A retinal tracking algorithm requires a reference image to compute the geometrical transformation between the reference and moving images. The tracking algorithm for auto-alignment and auto-capture can be used in different scenarios.

For example, in a first scenario (scenario-1) may be if A reference image of the retina from previous doctor's office visits for a patient fixation is available. In this case, the reference image can be used to align and trigger an auto-capture when the eye is stable (with no motion or minimal motion), and a sequence of retinal images are tracked robustly. A second scenario (scenario-2) may be if a retinal image quality algorithm detects a reference image during initial alignment (by the operator or automatic). In this case, the detected reference image by the image quality algorithm can be used in a similar manner as in scenario-1 to align and trigger an auto-capture. A third scenario (scenario-3) may be if a reference image from a previous visit and a retinal image quality algorithm are not available. In this case, the algorithm may track a sequence of images starting from the last image in a previous sequence as the reference image. The algorithm may repeat this process until consecutive sequences of images are tracked continuously and robustly, which can trigger an auto-capture.

In this present embodiment, auto-alignment and auto-capture approaches using the methods described in the above three scenarios using a retinal tracking system are described. The basic idea is to use the tracking algorithm to evaluate the fundus image (e.g., determine if the fundus image is a good quality retinal image for a given fixation) and eye motion relative to the fixation position during alignment. An auto-capture is triggered if the eye motion is minimal at the fixation position. Additionally, tracking outputs (e.g. xy translation and rotation relative to the fixation position) can also be used for automated alignment in a motorized system by moving the hardware components such as chin-rest or head-rest, ocular lens, etc.

As mentioned above, IR preview images (90-degree FOV) are typically used for patient alignment. These images along with an IR tracking algorithm, such as described above or other known IR tracking algorithm, can be used to determine if the images are trackable continuously and robustly using a reference image. The following are some embodiments suitable for use with the above-three mentioned different scenarios.

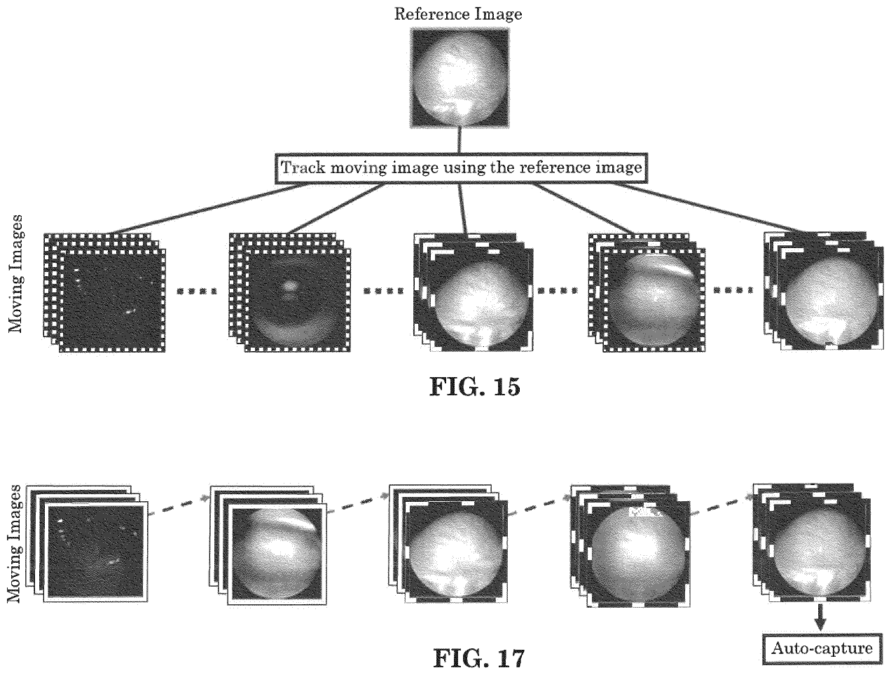

FIG. 15 illustrates scenario-1, wherein a reference image of the retina from previous visits for a patient fixation is available. In this scenario, the alignment and auto-capture are a simple problem as the reference image is known for a given fixation. FIG. 15 shows that each moving image is tracked using the reference image. A dotted-outline frame is a not trackable image and dashed-outline frame is a trackable image. The quality of tracking determines if the image is in a correct fixation and has a good quality. The quality of tracking may be measured using the tracking outputs such as tracking error, landmark distribution, number of landmarks, xy-translation, and rotation of the moving image relative to the reference image, etc. (such as described above). The tracking outputs can be also used for automated alignment in a motorized system by moving the hardware components such as chin-rest or head-rest, ocular lens, etc.

An auto-capture can be triggered if a predefined number N of consecutive moving images are robustly trackable (e.g., with a predefined confidence or quality measure). This indicates that the patient's eye has minimal motion and the fixation is correct. Tracking outputs can be also used to guide the operator or the patient (graphically or using sound/verbal/text) for better alignment.

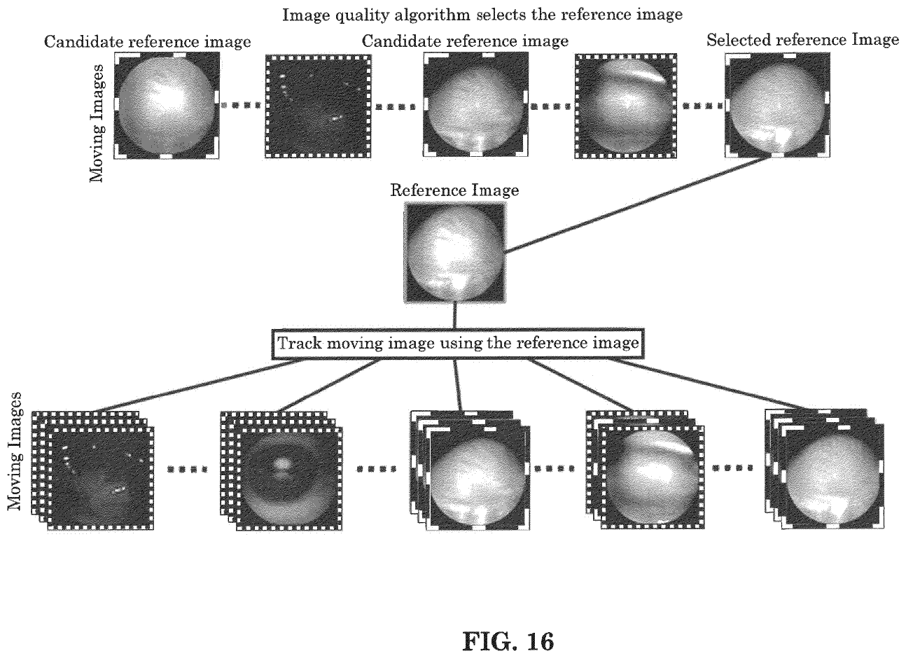

FIG. 16 illustrates scenario-2, where a retinal image quality algorithm detects the reference image during initial alignment (by the operator or automatic). In this scenario, the reference image is detected using a suitable IR image quality algorithm from sequences of moving images during alignment. The operator performs the initial alignment to bring the retina in the desired field of view and fixation. Then, the IR image quality algorithm determines the quality of a sequence of moving images. A reference image is then selected from a set of reference image candidates. The best reference image is selected based on the image quality score. Once the reference image is selected, then an auto-capture or auto-alignment can be triggered as described above in reference to scenario-1.

In scenario-3, the reference image from previous visits and the retinal image quality algorithm are not available. In this scenario, the algorithm tracks a sequence of images starting from the last image in the previous sequence as the reference image (solid-white frames). FIG. 17 shows that the algorithm repeats this process until consecutive sequences of images are tracked (dashed-outline frames) continuously and robustly, which can trigger an auto-capture. In this approach, the operator may do the initial alignment to bring the retina into the desired field of view and fixation.

The number of images in a sequence depends on the tracking performance. For instance, if the tracking is not possible, a new sequence can be started with a new reference image from the last image in the previous sequence.

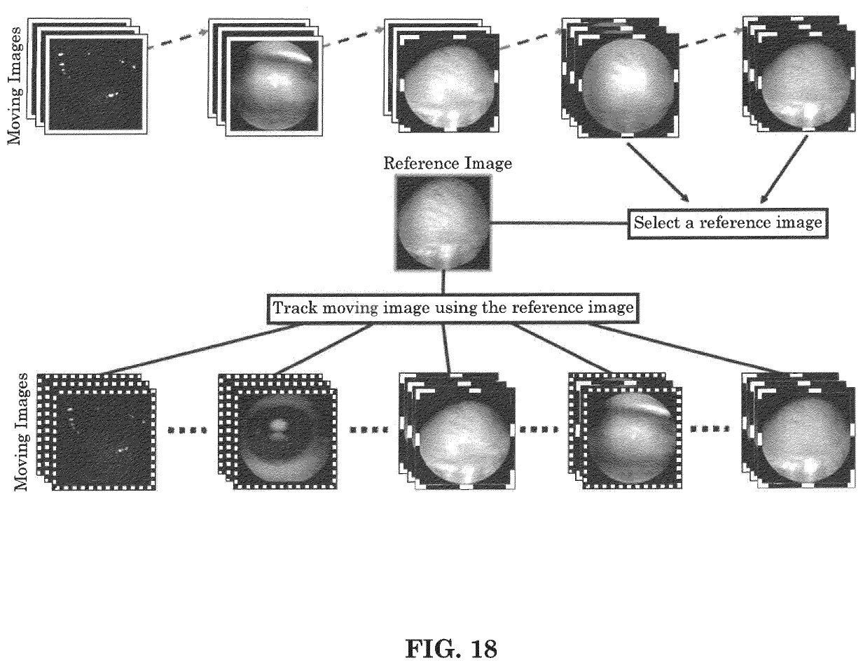

FIG. 18 illustrates an alternative solution for scenario-3. This approach may to select a reference image from the consecutive sequences of images that were tracked continuously and robustly. Once the reference image is selected, then an auto-capture or auto-alignment can be triggered as in the method of scenario-1.

The above described imaging tracking applications may be used to extract various statistics for identifying various characteristic issues that affect tracking. For example, images sequences used for tracking may be analyzed to determine if the images are characteristic of systemic movement, random movement, or good fixation. An ophthalmic system using the present invention may then inform the system operator or a patient of the possible issue affecting tracking, and provide suggested solutions.

Various types of artifacts in OCT may impact the diagnostics of eye diseases. Off-centering and motion artifacts are among important artifacts. Off-center artifact is due to a fixation error, causing the displacement of the analysis grid on topographic map for a specific disease type. Off-center artifact happens mostly with subjects with poor attention, poor vision or eccentric fixation. Even though the patient is asked to fixate, involuntary eye motions still happen, with different strengths in different directions at the time of alignment and acquisition.

The motion artifacts are due to ocular saccades, change of head position or due to respiratory movements. Motion artifacts can be overcome by eye a tracking system. However, eye tracking systems generally cannot handle saccade motion or a patient with poor attention or poor vision. In these cases, the scan cannot be fully completed to the end.

To improve patient fixation and reduce distraction (specifically for longer scan time), particularly in patients with poor attention or poor vision, eye motion analysis during alignment and acquisition could be a helpful tool to notify the operator as well as the patient that more careful attention is needed for a better fixation or eye motion control. For instance, a visual notification for an operator and a sound notification for a patient may be provided, and this may lead to a more successful scan. The operator could adjust the hardware component according to the motion analysis outputs. The patient could be guided to the fixation target until the scan is finished.

In the present embodiment, a method for eye motion analysis is described. The basic idea is to use the retinal tracking outputs for a real-time analysis or a post-acquisition analysis to generate a set of messages including sound messages which can notify the operator as well as the patient during alignment and acquisition about the state of fixation and eye motion. Providing motion analysis results after acquisition could help the operator to understand the reason for poor scan quality so that the operator could take appropriate action which may lead to successful scans.

The eye motion analysis can be helpful for the following:

1) Self-alignment: the patient can receive an instruction from the device to align.
2) Automated acquisition: during acquisition patient can be notified about the fixation change or large motion. Fixation in the same location is important for small scan field of views.
3) When fixation target is not available, a message (e.g. in form of sound) could keep the patient fixated.
4) The eye motion analysis results can be used for a post-processing algorithm to resolve the residual motion.

The above-described real-time retinal tracking method for off-centered fixation using infrared-reflectance (IR) images was tested in a proof-of-concept application. As is explained above, OCT acquisition systems rely on robust and real-time retinal tracking methods to capture reliable OCT images for visualization and further analysis. Tracking the retina with off-centered fixation can be a challenge due to a lack of adequate rich anatomical features in the images. The presently proposed robust and real-time retinal tracking algorithm finds at least one anatomical feature with high contrast as a reference point (RP) to improve the tracking performance.

In the present example, as is discussed above, a real-time keypoint (KP) based registration between a reference and a moving image calculates the xy-translation and rotation as the tracking parameters. The present tracking method relies on a unique RP and a set of reference image KPs extracted from the reference image. The location of the RP in the reference image is robustly detected using a fast image saliency method. Any suitable saliency method known the art may be used. Examples saliency may be found in: (1) X. Hou and L. Zhang, “Saliency Detection: A Spectral Residual Approach,” in CVPR, 2007; (2) C. Guo, Q. Ma, and L. Zhang, “Spatio-temporal saliency detection using phase spectrum of quaternion fourier transform,” in CVPR, 2008; and (3) B. Schauerte, B. Kuhn, K. Kroschel, R. Stiefelhagen, “Multimodal Saliency-based Attention for Object-based Scene Analysis,” in IROS, 2011.

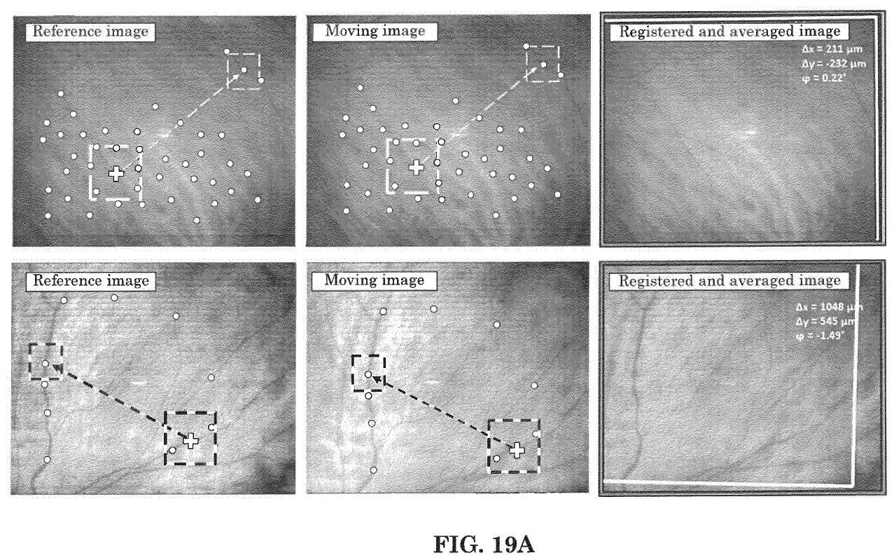

FIG. 19A shows two examples for small (top) and normal (bottom) pupil acquisition modes. RP (+) is detected by use of the fast image saliency algorithm. Keypoints (white circles) are detected relative to the RP location. The RP location in moving images is detected by template-matching using RP template extracted from the reference image. Each reference KP template and its relative distance to the RP are used to search for the corresponding moving KP with the same distance from the moving image RP location, as is illustrated by the dashed arrow.

In the present example implementation, the tracking parameters were calculated from a subset of KP correspondences with high confidence. Prototype software was used to collect sequences of IR images (11.52×9.36 mm with a pixel size of 15 μm/pixel and 50 Hz frame rate) from a CLARUS 500 (ZEISS, Dublin, Calif.). The registered images were displayed in a single image to visualize the registration (e.g., the right-most images in FIG. 19A). The mean distance error between the registered moving and reference KPs for each moving image were calculated as the registration error.

The statistics of registration error, number of KPs and eye motion were reported. FIG. 19B shows statistics for the registration error, eye motion and number of keypoints for a total number of 29,529 images from 45 sequences of images. The 45 sequences, with an average of 650 images each, were collected from one or both eyes of ten subjects/patients. The patients' fixations were off-centered. The average registration error of 15.3±2.7 μm indicates that accurate tracking in OCT domain with A-scan spacing greater than 15 μm is possible. The execution time of the tracking was measured as 15 ms on average using Intel i7-8850H CPU, 2.6 GHz, 32 GB RAM. Thus, the present implementation demonstrates the robustness of the present tracking algorithm based on a real-time retinal tracking method using IR fundus images. This tool could be an important part of any OCT image acquisition system.

Most eye tracking based analysis aim to identify and analyze patterns of visual attention of individuals as they perform specific tasks such as reading, searching, scanning an image, driving, etc. Anterior segment of the eye (such as pupil and iris) is used for eye motion analysis. The present approach uses retinal tracking outputs (eye motion parameters) for each frame of a Line-scan Ophthalmoscope (LSO) or infrared-reflectance (IR) fundus image. Recorded eye motion parameters (x,y, e.g., translation and rotation) in a time period can be used for a statistical analysis, which may include the statistical moment analysis of eye motion parameters. Future eye motion can be predicted using time series analysis such as Kalman filtering and particle filter. The present system may also generate massages using statistical and time series analysis to notify the operator and patient.

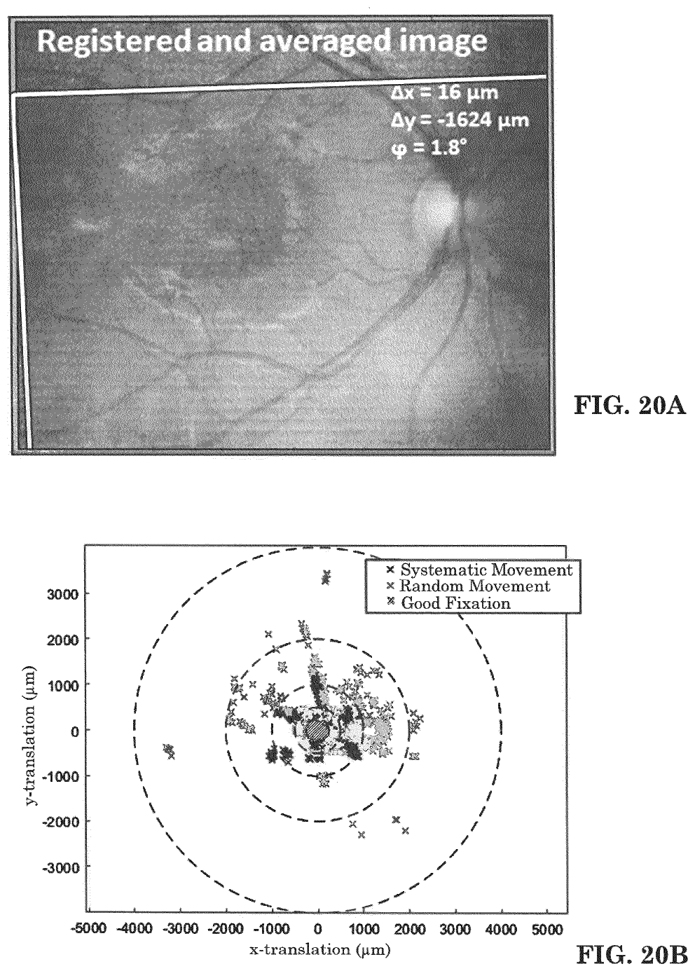

In the present invention, eye motion analysis can be used during and/or after acquisition. A retinal tracking algorithm using LSO or IR images can be used to calculate the eye motion parameters such as x and y translation and rotation. The motion parameters are calculated relative to a reference image, which is captured with the initial fixation or using any of the above-described methods. FIG. 20A illustrates the motion of a current image (white boarder) relative to a reference image (gray boarder) with eye motion parameters of Ax, Ay, and rotation co relative to the reference image. The current image was registered to the reference image followed by averaging of two images.

For each fundus image, the eye motion parameters are recorded, which can be used for a statistical analysis in a time period. Example of statistical analysis includes the statistical moment analysis of eye motion parameters. A time series analysis can be used for future eye motion prediction. The predication algorithms include Kalman filtering and particle filter. An informative massage can be generated using statistical and time series analysis to notify the operator and patient for an action.

In an embodiment with motion analysis during alignment and acquisition, time series analysis for eye motion prediction (next position and velocity) can warn the patient if he/she is drifting away from an initial fixation position.

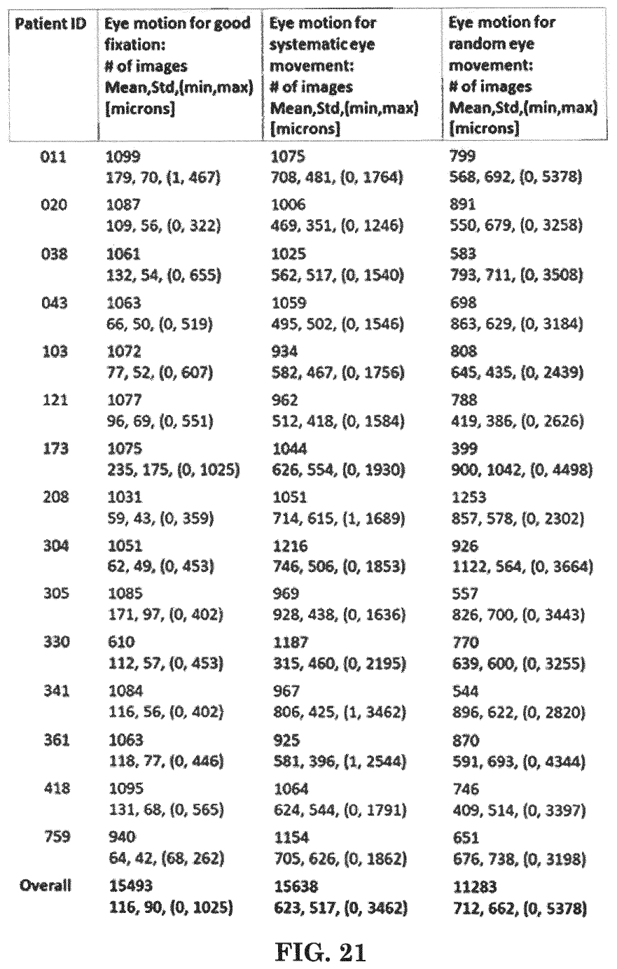

In an embodiment with motion analysis after acquisition, statistical analysis may be applied after termination of a current acquisition (irrespective of whether the acquisition was successful or failed). One example of the statistical analysis includes the overall fixation offset (mean value of xy motion) from the initial fixation position and the distribution (standard deviation) of eye motion as a measure of eye motion severity during an acquisition. FIG. 20B shows an example from three different patients, one good fixation, another with systematic eye movement, and the third with random eye movement. The eye motion calculation may be applied to an IR image relative to a reference image with initial fixation. FIG. 21 provides a table that shows the statistics of eye motion for 15 patients. The mean value indicates the overall fixation offset from the initial fixation position. The standard deviation indicates a measure of eye motion within an acquisition. Scans containing systematic or random eye movement show significantly greater mean and standard deviation, as compared to scans with good fixation, which may be used as an indicator for poor fixation. Significantly greater mean or standard deviation may be defined as 116 and 90 micron, respectively, for this study. The eye and fixation analysis can highlight its use as feedback for the operator or the patient by providing informative messages for reduced motion in OCT image acquisition, which is important for any subsequent data processing.

Hereinafter is provided a description of various hardware and architectures suitable for the present invention.

### Fundus Imaging System

Two categories of imaging systems used to image the fundus are flood illumination imaging systems (or flood illumination imagers) and scan illumination imaging systems (or scan imagers). Flood illumination imagers flood with light an entire field of view (FOV) of interest of a specimen at the same time, such as by use of a flash lamp, and capture a full-frame image of the specimen (e.g., the fundus) with a full-frame camera (e.g., a camera having a two-dimensional (2D) photo sensor array of sufficient size to capture the desired FOV, as a whole). For example, a flood illumination fundus imager would flood the fundus of an eye with light, and capture a full-frame image of the fundus in a single image capture sequence of the camera. A scan imager provides a scan beam that is scanned across a subject, e.g., an eye, and the scan beam is imaged at different scan positions as it is scanned across the subject creating a series of image-segments that may be reconstructed, e.g., montaged, to create a composite image of the desired FOV. The scan beam could be a point, a line, or a two-dimensional area such a slit or broad line. Examples of fundus imagers are provided in U.S. Pat. Nos. 8,967,806 and 8,998,411.

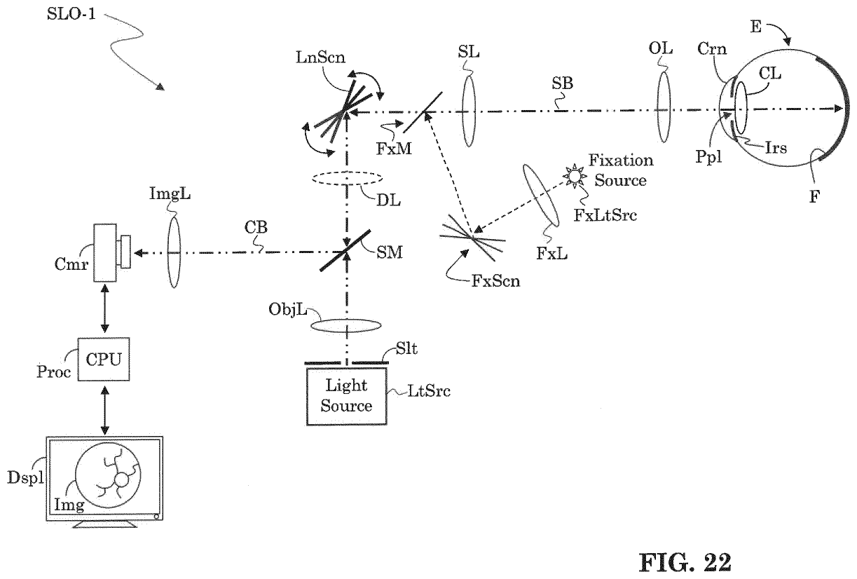

FIG. 22 illustrates an example of a slit scanning ophthalmic system SLO-1 for imaging a fundus F, which is the interior surface of an eye E opposite the eye lens (or crystalline lens) CL and may include the retina, optic disc, macula, fovea, and posterior pole. In the present example, the imaging system is in a so-called “scan-descan” configuration, wherein a scanning line beam SB traverses the optical components of the eye E (including the cornea Cm, iris Irs, pupil Ppl, and crystalline lens CL) to be scanned across the fundus F. In the case of a flood fundus imager, no scanner is needed, and the light is applied across the entire, desired field of view (FOV) at once. Other scanning configurations are known in the art, and the specific scanning configuration is not critical to the present invention. As depicted, the imaging system includes one or more light sources LtSrc, preferably a multi-color LED system or a laser system in which the etendue has been suitably adjusted. An optional slit Slt (adjustable or static) is positioned in front of the light source LtSrc and may be used to adjust the width of the scanning line beam SB. Additionally, slit Slt may remain static during imaging or may be adjusted to different widths to allow for different confocality levels and different applications either for a particular scan or during the scan for use in suppressing reflexes. An optional objective lens ObjL may be placed in front of the slit Slt. The objective lens ObjL can be any one of state-of-the-art lenses including but not limited to refractive, diffractive, reflective, or hybrid lenses/systems. The light from slit Slt passes through a pupil splitting mirror SM and is directed towards a scanner LnScn. It is desirable to bring the scanning plane and the pupil plane as near together as possible to reduce vignetting in the system. Optional optics DL may be included to manipulate the optical distance between the images of the two components. Pupil splitting mirror SM may pass an illumination beam from light source LtSrc to scanner LnScn, and reflect a detection beam from scanner LnScn (e.g., reflected light returning from eye E) toward a camera Cmr. A task of the pupil splitting mirror SM is to split the illumination and detection beams and to aid in the suppression of system reflexes. The scanner LnScn could be a rotating galvo scanner or other types of scanners (e.g., piezo or voice coil, micro-electromechanical system (MEMS) scanners, electro-optical deflectors, and/or rotating polygon scanners). Depending on whether the pupil splitting is done before or after the scanner LnScn, the scanning could be broken into two steps wherein one scanner is in an illumination path and a separate scanner is in a detection path. Specific pupil splitting arrangements are described in detail in U.S. Pat. No. 9,456,746, which is herein incorporated in its entirety by reference.

From the scanner LnScn, the illumination beam passes through one or more optics, in this case a scanning lens SL and an ophthalmic or ocular lens OL, that allow for the pupil of the eye E to be imaged to an image pupil of the system. Generally, the scan lens SL receives a scanning illumination beam from the scanner LnScn at any of multiple scan angles (incident angles), and produces scanning line beam SB with a substantially flat surface focal plane (e.g., a collimated light path). Ophthalmic lens OL may then focus the scanning line beam SB onto an object to be imaged. In the present example, ophthalmic lens OL focuses the scanning line beam SB onto the fundus F (or retina) of eye E to image the fundus. In this manner, scanning line beam SB creates a traversing scan line that travels across the fundus F. One possible configuration for these optics is a Kepler type telescope wherein the distance between the two lenses is selected to create an approximately telecentric intermediate fundus image (4-f configuration). The ophthalmic lens OL could be a single lens, an achromatic lens, or an arrangement of different lenses. All lenses could be refractive, diffractive, reflective or hybrid as known to one skilled in the art. The focal length(s) of the ophthalmic lens OL, scan lens SL and the size and/or form of the pupil splitting mirror SM and scanner LnScn could be different depending on the desired field of view (FOV), and so an arrangement in which multiple components can be switched in and out of the beam path, for example by using a flip in optic, a motorized wheel, or a detachable optical element, depending on the field of view can be envisioned. Since the field of view change results in a different beam size on the pupil, the pupil splitting can also be changed in conjunction with the change to the FOV. For example, a 45° to 60° field of view is a typical, or standard, FOV for fundus cameras. Higher fields of view, e.g., a widefield FOV, of 60°-120°, or more, may also be feasible. A widefield FOV may be desired for a combination of the Broad-Line Fundus Imager (BLFI) with another imaging modalities such as optical coherence tomography (OCT). The upper limit for the field of view may be determined by the accessible working distance in combination with the physiological conditions around the human eye. Because a typical human retina has a FOV of 140° horizontal and 80°-100° vertical, it may be desirable to have an asymmetrical field of view for the highest possible FOV on the system.

The scanning line beam SB passes through the pupil Ppl of the eye E and is directed towards the retinal, or fundus, surface F. The scanner LnScn1 adjusts the location of the light on the retina, or fundus, F such that a range of transverse locations on the eye E are illuminated. Reflected or scattered light (or emitted light in the case of fluorescence imaging) is directed back along as similar path as the illumination to define a collection beam CB on a detection path to camera Cmr.

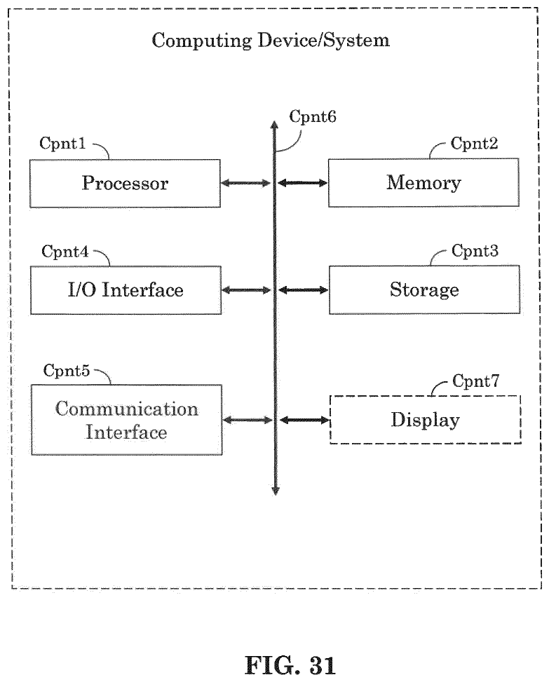

In the “scan-descan” configuration of the present, exemplary slit scanning ophthalmic system SLO-1, light returning from the eye E is “descanned” by scanner LnScn on its way to pupil splitting mirror SM. That is, scanner LnScn scans the illumination beam from pupil splitting mirror SM to define the scanning illumination beam SB across eye E, but since scanner LnScn also receives returning light from eye E at the same scan position, scanner LnScn has the effect of descanning the returning light (e.g., cancelling the scanning action) to define a non-scanning (e.g., steady or stationary) collection beam from scanner LnScn to pupil splitting mirror SM, which folds the collection beam toward camera Cmr. At the pupil splitting mirror SM, the reflected light (or emitted light in the case of fluorescence imaging) is separated from the illumination light onto the detection path directed towards camera Cmr, which may be a digital camera having a photo sensor to capture an image. An imaging (e.g., objective) lens ImgL may be positioned in the detection path to image the fundus to the camera Cmr. As is the case for objective lens ObjL, imaging lens ImgL may be any type of lens known in the art (e.g., refractive, diffractive, reflective or hybrid lens). Additional operational details, in particular, ways to reduce artifacts in images, are described in PCT Publication No. WO2016/124644, the contents of which are herein incorporated in their entirety by reference. The camera Cmr captures the received image, e.g., it creates an image file, which can be further processed by one or more (electronic) processors or computing devices (e.g., the computer system of FIG. 31). Thus, the collection beam (returning from all scan positions of the scanning line beam SB) is collected by the camera Cmr, and a full-frame image Img may be constructed from a composite of the individually captured collection beams, such as by montaging. However, other scanning configuration are also contemplated, including ones where the illumination beam is scanned across the eye E and the collection beam is scanned across a photo sensor array of the camera. PCT Publication WO 2012/059236 and US Patent Publication No. 2015/0131050, herein incorporated by reference, describe several embodiments of slit scanning ophthalmoscopes including various designs where the returning light is swept across the camera's photo sensor array and where the returning light is not swept across the camera's photo sensor array.

In the present example, the camera Cmr is connected to a processor (e.g., processing module) Proc and a display (e.g., displaying module, computer screen, electronic screen, etc.) Dspl, both of which can be part of the image system itself, or may be part of separate, dedicated processing and/or displaying unit(s), such as a computer system wherein data is passed from the camera Cmr to the computer system over a cable or computer network including wireless networks. The display and processor can be an all in one unit. The display can be a traditional electronic display/screen or of the touch screen type and can include a user interface for displaying information to and receiving information from an instrument operator, or user. The user can interact with the display using any type of user input device as known in the art including, but not limited to, mouse, knobs, buttons, pointer, and touch screen.

It may be desirable for a patient's gaze to remain fixed while imaging is carried out. One way to achieve this is to provide a fixation target that the patient can be directed to stare at. Fixation targets can be internal or external to the instrument depending on what area of the eye is to be imaged. One embodiment of an internal fixation target is shown in FIG. 22. In addition to the primary light source LtSrc used for imaging, a second optional light source FxLtSrc, such as one or more LEDs, can be positioned such that a light pattern is imaged to the retina using lens FxL, scanning element FxScn and reflector/mirror FxM. Fixation scanner FxScn can move the position of the light pattern and reflector FxM directs the light pattern from fixation scanner FxScn to the fundus F of eye E. Preferably, fixation scanner FxScn is position such that it is located at the pupil plane of the system so that the light pattern on the retina/fundus can be moved depending on the desired fixation location.

Slit-scanning ophthalmoscope systems are capable of operating in different imaging modes depending on the light source and wavelength selective filtering elements employed. True color reflectance imaging (imaging similar to that observed by the clinician when examining the eye using a hand-held or slit lamp ophthalmoscope) can be achieved when imaging the eye with a sequence of colored LEDs (red, blue, and green). Images of each color can be built up in steps with each LED turned on at each scanning position or each color image can be taken in its entirety separately. The three, color images can be combined to display the true color image, or they can be displayed individually to highlight different features of the retina. The red channel best highlights the choroid, the green channel highlights the retina, and the blue channel highlights the anterior retinal layers. Additionally, light at specific frequencies (e.g., individual colored LEDs or lasers) can be used to excite different fluorophores in the eye (e.g., autofluorescence) and the resulting fluorescence can be detected by filtering out the excitation wavelength.

The fundus imaging system can also provide an infrared reflectance image, such as by using an infrared laser (or other infrared light source). The infrared (IR) mode is advantageous in that the eye is not sensitive to the IR wavelengths. This may permit a user to continuously take images without disturbing the eye (e.g., in a preview/alignment mode) to aid the user during alignment of the instrument. Also, the IR wavelengths have increased penetration through tissue and may provide improved visualization of choroidal structures. In addition, fluorescein angiography (FA) and indocyanine green (ICG) angiography imaging can be accomplished by collecting images after a fluorescent dye has been injected into the subject's bloodstream. For example, in FA (and/or ICG) a series of time-lapse images may be captured after injecting a light-reactive dye (e.g., fluorescent dye) into a subject's bloodstream. It is noted that care must be taken since the fluorescent dye may lead to a life-threatening allergic reaction in a portion of the population. High contrast, greyscale images are captured using specific light frequencies selected to excite the dye. As the dye flows through the eye, various portions of the eye are made to glow brightly (e.g., fluoresce), making it possible to discern the progress of the dye, and hence the blood flow, through the eye.

### Optical Coherence Tomography Imaging System

Generally, optical coherence tomography (OCT) uses low-coherence light to produce two-dimensional (2D) and three-dimensional (3D) internal views of biological tissue. OCT enables in vivo imaging of retinal structures. OCT angiography (OCTA) produces flow information, such as vascular flow from within the retina. Examples of OCT systems are provided in U.S. Pat. Nos. 6,741,359 and 9,706,915, and examples of an OCTA systems may be found in U.S. Pat. Nos. 9,700,206 and 9,759,544, all of which are herein incorporated in their entirety by reference. An exemplary OCT/OCTA system is provided herein.

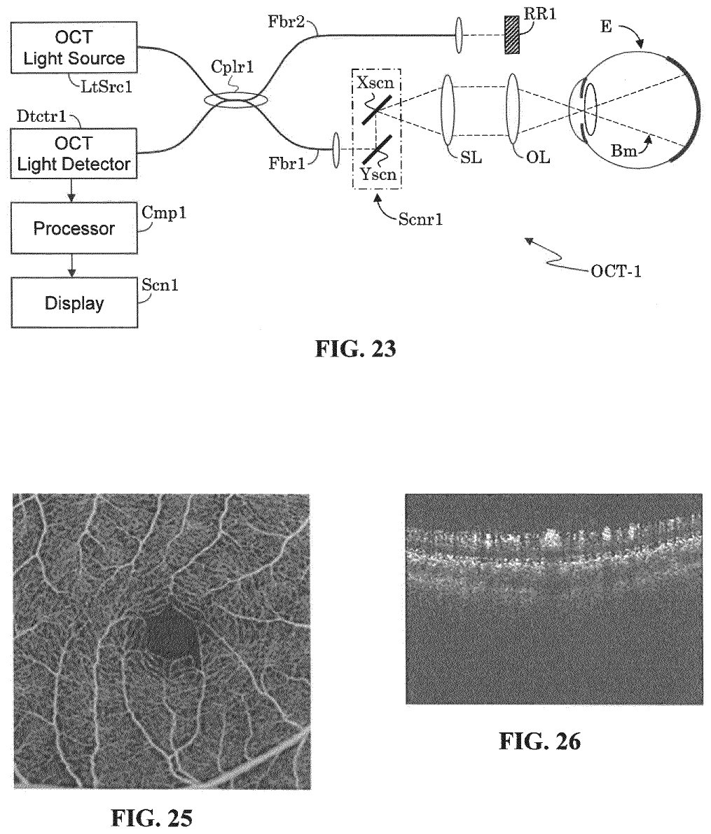

FIG. 23 illustrates a generalized frequency domain optical coherence tomography (FD-OCT) system used to collect 3D image data of the eye suitable for use with the present invention. An FD-OCT system OCT_1 includes a light source, LtSrc1. Typical light sources include, but are not limited to, broadband light sources with short temporal coherence lengths or swept laser sources. A beam of light from light source LtSrc1 is routed, typically by optical fiber Fbr1, to illuminate a sample, e.g., eye E; a typical sample being tissues in the human eye. The light source LrSrc1 may, for example, be a broadband light source with short temporal coherence length in the case of spectral domain OCT (SD-OCT) or a wavelength tunable laser source in the case of swept source OCT (SS-OCT). The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL may receive the beam of light from the scanner Scnr1 at multiple incident angles and produces substantially collimated light, ophthalmic lens OL may then focus onto the sample. The present example illustrates a scan beam that needs to be scanned in two lateral directions (e.g., in x and y directions on a Cartesian plane) to scan a desired field of view (FOV). An example of this would be a point-field OCT, which uses a point-field beam to scan across a sample. Consequently, scanner Scnr1 is illustratively shown to include two sub-scanner: a first sub-scanner Xscn for scanning the point-field beam across the sample in a first direction (e.g., a horizontal x-direction); and a second sub-scanner Yscn for scanning the point-field beam on the sample in traversing second direction (e.g., a vertical y-direction). If the scan beam were a line-field beam (e.g., a line-field OCT), which may sample an entire line-portion of the sample at a time, then only one scanner may be needed to scan the line-field beam across the sample to span the desired FOV. If the scan beam were a full-field beam (e.g., a full-field OCT), no scanner may be needed, and the full-field light beam may be applied across the entire, desired FOV at once.

Irrespective of the type of beam used, light scattered from the sample (e.g., sample light) is collected. In the present example, scattered light returning from the sample is collected into the same optical fiber Fbr1 used to route the light for illumination. Reference light derived from the same light source LtSrc1 travels a separate path, in this case involving optical fiber Fbr2 and retro-reflector RR1 with an adjustable optical delay. Those skilled in the art will recognize that a transmissive reference path can also be used and that the adjustable delay could be placed in the sample or reference arm of the interferometer. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, those skilled in the art will recognize that various designs of interferometers can be used for balanced or unbalanced detection of the interference signal. The output from the detector Dtctr1 is supplied to a processor (e.g., internal or external computing device) Cmp1 that converts the observed interference into depth information of the sample. The depth information may be stored in a memory associated with the processor Cmp1 and/or displayed on a display (e.g., computer/electronic display/screen) Scn1. The processing and storing functions may be localized within the OCT instrument, or functions may be offloaded onto (e.g., performed on) an external processor (e.g., an external computing device), to which the collected data may be transferred. An example of a computing device (or computer system) is shown in FIG. 31. This unit could be dedicated to data processing or perform other tasks which are quite general and not dedicated to the OCT device. The processor (computing device) Cmp1 may include, for example, a field-programmable gate array (FPGA), a digital signal processor (DSP), an application specific integrated circuit (ASIC), a graphics processing unit (GPU), a system on chip (SoC), a central processing unit (CPU), a general purpose graphics processing unit (GPGPU), or a combination thereof, that may performs some, or the entire, processing steps in a serial and/or parallelized fashion with one or more host processors and/or one or more external computing devices.

The sample and reference arms in the interferometer could consist of bulk-optics, fiber-optics, or hybrid bulk-optic systems and could have different architectures such as Michelson, Mach-Zehnder or common-path based designs as would be known by those skilled in the art. Light beam as used herein should be interpreted as any carefully directed light path. Instead of mechanically scanning the beam, a field of light can illuminate a one or two-dimensional area of the retina to generate the OCT data (see for example, U.S. Pat. No. 9,332,902; D. Hillmann et al, “Holoscopy—Holographic Optical Coherence Tomography,” Optics Letters, 36(13): 2390 2011; Y. Nakamura, et al, “High-Speed Three Dimensional Human Retinal Imaging by Line Field Spectral Domain Optical Coherence Tomography,” Optics Express, 15(12):7103 2007; Blazkiewicz et al, “Signal-To-Noise Ratio Study of Full-Field Fourier-Domain Optical Coherence Tomography,” Applied Optics, 44(36):7722 (2005)). In time-domain systems, the reference arm needs to have a tunable optical delay to generate interference. Balanced detection systems are typically used in TD-OCT and SS-OCT systems, while spectrometers are used at the detection port for SD-OCT systems. The invention described herein could be applied to any type of OCT system. Various aspects of the invention could apply to any type of OCT system or other types of ophthalmic diagnostic systems and/or multiple ophthalmic diagnostic systems including but not limited to fundus imaging systems, visual field test devices, and scanning laser polarimeters.

In Fourier Domain optical coherence tomography (FD-OCT), each measurement is the real-valued spectral interferogram (Sj(k)). The real-valued spectral data typically goes through several post-processing steps including background subtraction, dispersion correction, etc. The Fourier transform of the processed interferogram, results in a complex valued OCT signal output Aj(z)=|Aj|ei φ. The absolute value of this complex OCT signal, |Aj|, reveals the profile of scattering intensities at different path lengths, and therefore scattering as a function of depth (z-direction) in the sample. Similarly, the phase, φj can also be extracted from the complex valued OCT signal. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. The term “cluster scan” may refer to a single unit or block of data generated by repeated acquisitions at the same (or substantially the same) location (or region) for the purposes of analyzing motion contrast, which may be used to identify blood flow. A cluster scan can consist of multiple A-scans or B-scans collected with relatively short time separations at approximately the same location(s) on the sample. Since the scans in a cluster scan are of the same region, static structures remain relatively unchanged from scan to scan within the cluster scan, whereas motion contrast between the scans that meets predefined criteria may be identified as blood flow.

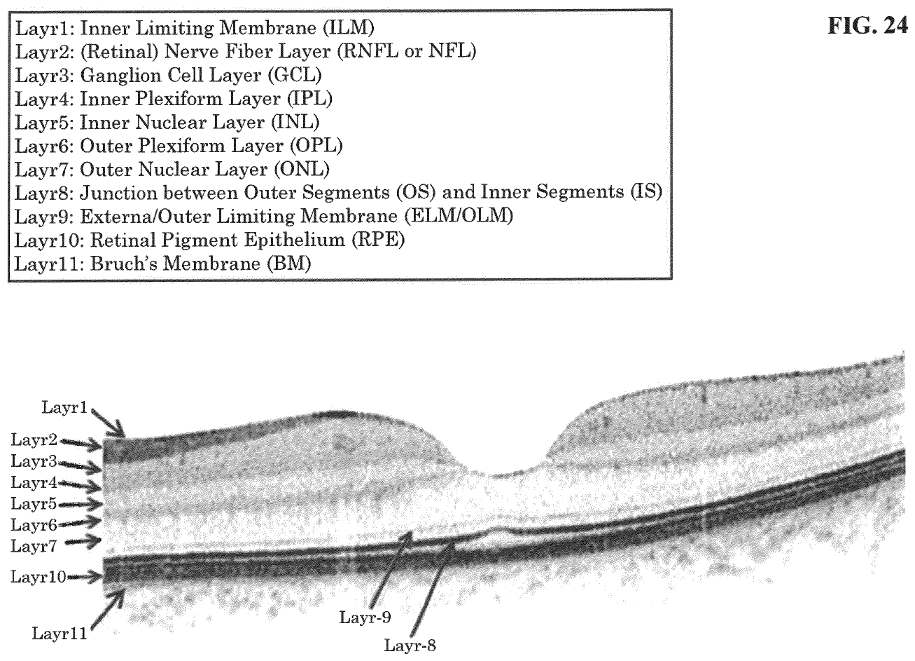

A variety of ways to create B-scans are known in the art including but not limited to: along the horizontal or x-direction, along the vertical or y-direction, along the diagonal of x and y, or in a circular or spiral pattern. B-scans may be in the x-z dimensions but may be any cross-sectional image that includes the z-dimension. An example OCT B-scan image of a normal retina of a human eye is illustrated in FIG. 24. An OCT B-scan of the retinal provides a view of the structure of retinal tissue. For illustration purposes, FIG. 24 identifies various canonical retinal layers and layer boundaries. The identified retinal boundary layers include (from top to bottom): the inner limiting membrane (ILM) Lyer1, the retinal nerve fiber layer (RNFL or NFL) Layr2, the ganglion cell layer (GCL) Layr3, the inner plexiform layer (IPL) Layr4, the inner nuclear layer (INL) Layr5, the outer plexiform layer (OPL) Layr6, the outer nuclear layer (ONL) Layr7, the junction between the outer segments (OS) and inner segments (IS) (indicated by reference character Layr8) of the photoreceptors, the external or outer limiting membrane (ELM or OLM) Layr9, the retinal pigment epithelium (RPE) Layr10, and the Bruch's membrane (BM) Layr11.

In OCT Angiography, or Functional OCT, analysis algorithms may be applied to OCT data collected at the same, or approximately the same, sample locations on a sample at different times (e.g., a cluster scan) to analyze motion or flow (see for example US Patent Publication Nos. 2005/0171438, 2012/0307014, 2010/0027857, 2012/0277579 and U.S. Pat. No. 6,549,801, all of which are herein incorporated in their entirety by reference). An OCT system may use any one of a number of OCT angiography processing algorithms (e.g., motion contrast algorithms) to identify blood flow. For example, motion contrast algorithms can be applied to the intensity information derived from the image data (intensity-based algorithm), the phase information from the image data (phase-based algorithm), or the complex image data (complex-based algorithm). An en face image is a 2D projection of 3D OCT data (e.g., by averaging the intensity of each individual A-scan, such that each A-scan defines a pixel in the 2D projection). Similarly, an en face vasculature image is an image displaying motion contrast signal in which the data dimension corresponding to depth (e.g., z-direction along an A-scan) is displayed as a single representative value (e.g., a pixel in a 2D projection image), typically by summing or integrating all or an isolated portion of the data (see for example U.S. Pat. No. 7,301,644 herein incorporated in its entirety by reference). OCT systems that provide an angiography imaging functionality may be termed OCT angiography (OCTA) systems.

FIG. 25 shows an example of an en face vasculature image. After processing the data to highlight motion contrast using any of the motion contrast techniques known in the art, a range of pixels corresponding to a given tissue depth from the surface of internal limiting membrane (ILM) in retina, may be summed to generate the en face (e.g., frontal view) image of the vasculature. FIG. 26 shows an exemplary B-scan of a vasculature (OCTA) image. As illustrated, structural information may not be well-defined since blood flow may traverse multiple retinal layers making them less defined than in a structural OCT B-scan, as shown in FIG. 24. Nonetheless, OCTA provides a non-invasive technique for imaging the microvasculature of the retina and the choroid, which may be critical to diagnosing and/or monitoring various pathologies. For example, OCTA may be used to identify diabetic retinopathy by identifying microaneurysms, neovascular complexes, and quantifying foveal avascular zone and nonperfused areas. Moreover, OCTA has been shown to be in good agreement with fluorescein angiography (FA), a more traditional, but more evasive, technique requiring the injection of a dye to observe vascular flow in the retina. Additionally, in dry age-related macular degeneration, OCTA has been used to monitor a general decrease in choriocapillaris flow. Similarly in wet age-related macular degeneration, OCTA can provides a qualitative and quantitative analysis of choroidal neovascular membranes. OCTA has also been used to study vascular occlusions, e.g., evaluation of nonperfused areas and the integrity of superficial and deep plexus.

### Neural Networks

As discussed above, the present invention may use a neural network (NN) machine learning (ML) model. For the sake of completeness, a general discussion of neural networks is provided herein. The present invention may use any, singularly or in combination, of the below described neural network architecture(s). A neural network, or neural net, is a (nodal) network of interconnected neurons, where each neuron represents a node in the network. Groups of neurons may be arranged in layers, with the outputs of one layer feeding forward to a next layer in a multilayer perceptron (MLP) arrangement. MLP may be understood to be a feedforward neural network model that maps a set of input data onto a set of output data. FIG. 25 illustrates an example of a multilayer perceptron (MLP) neural network. Its structure may include multiple hidden (e.g., internal) layers HL1 to HLn that map an input layer InL (that receives a set of inputs (or vector input) in_1 to in_3) to an output layer OutL that produces a set of outputs (or vector output), e.g., out_1 and out_2. Each layer may have any given number of nodes, which are herein illustratively shown as circles within each layer. In the present example, the first hidden layer HL1 has two nodes, while hidden layers HL2, HL3, and HLn each have three nodes. Generally, the deeper the MLP (e.g., the greater the number of hidden layers in the MLP), the greater its capacity to learn. The input layer InL receives a vector input (illustratively shown as a three-dimensional vector consisting of in_1, in_2 and in_3), and may apply the received vector input to the first hidden layer HL1 in the sequence of hidden layers. An output layer OutL receives the output from the last hidden layer, e.g., HLn, in the multilayer model, processes its inputs, and produces a vector output result (illustratively shown as a two-dimensional vector consisting of out_1 and out_2).

Typically, each neuron (or node) produces a single output that is fed forward to neurons in the layer immediately following it. But each neuron in a hidden layer may receive multiple inputs, either from the input layer or from the outputs of neurons in an immediately preceding hidden layer. In general, each node may apply a function to its inputs to produce an output for that node. Nodes in hidden layers (e.g., learning layers) may apply the same function to their respective input(s) to produce their respective output(s). Some nodes, however, such as the nodes in the input layer InL receive only one input and may be passive, meaning that they simply relay the values of their single input to their output(s), e.g., they provide a copy of their input to their output(s), as illustratively shown by dotted arrows within the nodes of input layer InL.

For illustration purposes, FIG. 28 shows a simplified neural network consisting of an input layer InL′, a hidden layer HL1′, and an output layer OutL′. Input layer InL′ is shown having two input nodes i1 and i2 that respectively receive inputs Input_1 and Input_2 (e.g. the input nodes of layer InL′ receive an input vector of two dimensions). The input layer InL′ feeds forward to one hidden layer HL1′ having two nodes h1 and h2, which in turn feeds forward to an output layer OutL′ of two nodes o1 and o2. Interconnections, or links, between neurons (illustrative shown as solid arrows) have weights w1 to w8. Typically, except for the input layer, a node (neuron) may receive as input the outputs of nodes in its immediately preceding layer. Each node may calculate its output by multiplying each of its inputs by each input's corresponding interconnection weight, summing the products of it inputs, adding (or multiplying by) a constant defined by another weight or bias that may be associated with that particular node (e.g., node weights w9, w10, w11, w12 respectively corresponding to nodes h1, h2, o1, and o2), and then applying a non-linear function or logarithmic function to the result. The non-linear function may be termed an activation function or transfer function. Multiple activation functions are known the art, and selection of a specific activation function is not critical to the present discussion. It is noted, however, that operation of the ML model, or behavior of the neural net, is dependent upon weight values, which may be learned so that the neural network provides a desired output for a given input.

The neural net learns (e.g., is trained to determine) appropriate weight values to achieve a desired output for a given input during a training, or learning, stage. Before the neural net is trained, each weight may be individually assigned an initial (e.g., random and optionally non-zero) value, e.g. a random-number seed. Various methods of assigning initial weights are known in the art. The weights are then trained (optimized) so that for a given training vector input, the neural network produces an output close to a desired (predetermined) training vector output. For example, the weights may be incrementally adjusted in thousands of iterative cycles by a technique termed back-propagation. In each cycle of back-propagation, a training input (e.g., vector input or training input image/sample) is fed forward through the neural network to determine its actual output (e.g., vector output). An error for each output neuron, or output node, is then calculated based on the actual neuron output and a target training output for that neuron (e.g., a training output image/sample corresponding to the present training input image/sample). One then propagates back through the neural network (in a direction from the output layer back to the input layer) updating the weights based on how much effect each weight has on the overall error so that the output of the neural network moves closer to the desired training output. This cycle is then repeated until the actual output of the neural network is within an acceptable error range of the desired training output for the given training input. As it would be understood, each training input may require many back-propagation iterations before achieving a desired error range. Typically, an epoch refers to one back-propagation iteration (e.g., one forward pass and one backward pass) of all the training samples, such that training a neural network may require many epochs. Generally, the larger the training set, the better the performance of the trained ML model, so various data augmentation methods may be used to increase the size of the training set. For example, when the training set includes pairs of corresponding training input images and training output images, the training images may be divided into multiple corresponding image segments (or patches). Corresponding patches from a training input image and training output image may be paired to define multiple training patch pairs from one input/output image pair, which enlarges the training set. Training on large training sets, however, places high demands on computing resources, e.g. memory and data processing resources. Computing demands may be reduced by dividing a large training set into multiple mini-batches, where the mini-batch size defines the number of training samples in one forward/backward pass. In this case, and one epoch may include multiple mini-batches. Another issue is the possibility of a NN overfitting a training set such that its capacity to generalize from a specific input to a different input is reduced. Issues of overfitting may be mitigated by creating an ensemble of neural networks or by randomly dropping out nodes within a neural network during training, which effectively removes the dropped nodes from the neural network. Various dropout regulation methods, such as inverse dropout, are known in the art.

It is noted that the operation of a trained NN machine model is not a straight-forward algorithm of operational/analyzing steps. Indeed, when a trained NN machine model receives an input, the input is not analyzed in the traditional sense. Rather, irrespective of the subject or nature of the input (e.g., a vector defining a live image/scan or a vector defining some other entity, such as a demographic description or a record of activity) the input will be subjected to the same predefined architectural construct of the trained neural network (e.g., the same nodal/layer arrangement, trained weight and bias values, predefined convolution/deconvolution operations, activation functions, pooling operations, etc.), and it may not be clear how the trained network's architectural construct produces its output.

Furthermore, the values of the trained weights and biases are not deterministic and depend upon many factors, such as the amount of time the neural network is given for training (e.g., the number of epochs in training), the random starting values of the weights before training starts, the computer architecture of the machine on which the NN is trained, selection of training samples, distribution of the training samples among multiple mini-batches, choice of activation function(s), choice of error function(s) that modify the weights, and even if training is interrupted on one machine (e.g., having a first computer architecture) and completed on another machine (e.g., having a different computer architecture). The point is that the reasons why a trained ML model reaches certain outputs is not clear, and much research is currently ongoing to attempt to determine the factors on which a ML model bases its outputs. Therefore, the processing of a neural network on live data cannot be reduced to a simple algorithm of steps. Rather, its operation is dependent upon its training architecture, training sample sets, training sequence, and various circumstances in the training of the ML model.

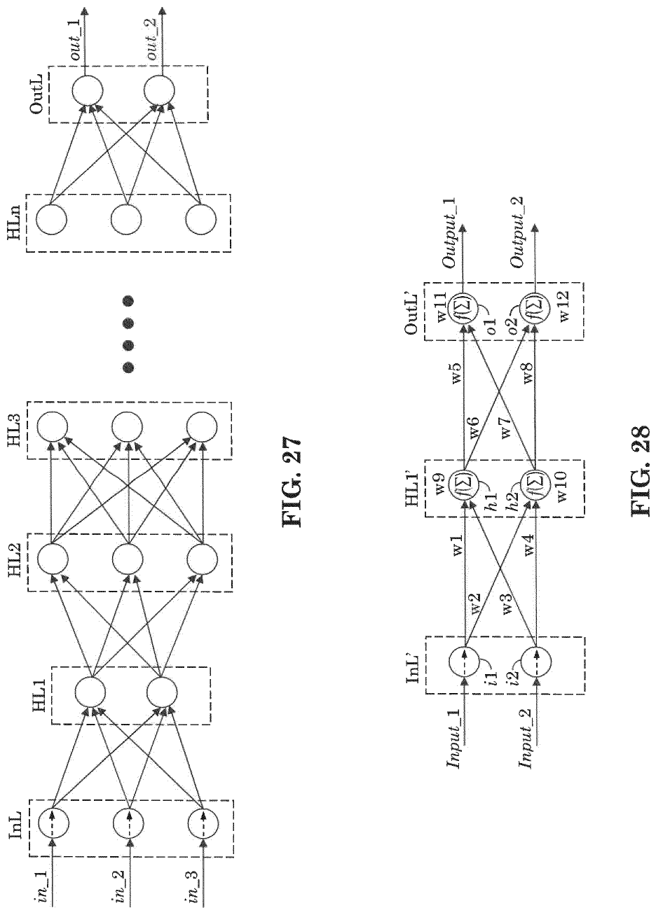

In summary, construction of a NN machine learning model may include a learning (or training) stage and a classification (or operational) stage. In the learning stage, the neural network may be trained for a specific purpose and may be provided with a set of training examples, including training (sample) inputs and training (sample) outputs, and optionally including a set of validation examples to test the progress of the training. During this learning process, various weights associated with nodes and node-interconnections in the neural network are incrementally adjusted in order to reduce an error between an actual output of the neural network and the desired training output. In this manner, a multi-layer feed-forward neural network (such as discussed above) may be made capable of approximating any measurable function to any desired degree of accuracy. The result of the learning stage is a (neural network) machine learning (ML) model that has been learned (e.g., trained). In the operational stage, a set of test inputs (or live inputs) may be submitted to the learned (trained) ML model, which may apply what it has learned to produce an output prediction based on the test inputs. Like the regular neural networks of FIGS. 27 and 28, convolutional neural networks (CNN) are also made up of neurons that have learnable weights and biases. Each neuron receives inputs, performs an operation (e.g., dot product), and is optionally followed by a non-linearity. The CNN, however, may receive raw image pixels at one end (e.g., the input end) and provide classification (or class) scores at the other end (e.g., the output end). Because CNNs expect an image as input, they are optimized for working with volumes (e.g., pixel height and width of an image, plus the depth of the image, e.g., color depth such as an RGB depth defined of three colors: red, green, and blue). For example, the layers of a CNN may be optimized for neurons arranged in 3 dimensions. The neurons in a CNN layer may also be connected to a small region of the layer before it, instead of all of the neurons in a fully-connected NN. The final output layer of a CNN may reduce a full image into a single vector (classification) arranged along the depth dimension.

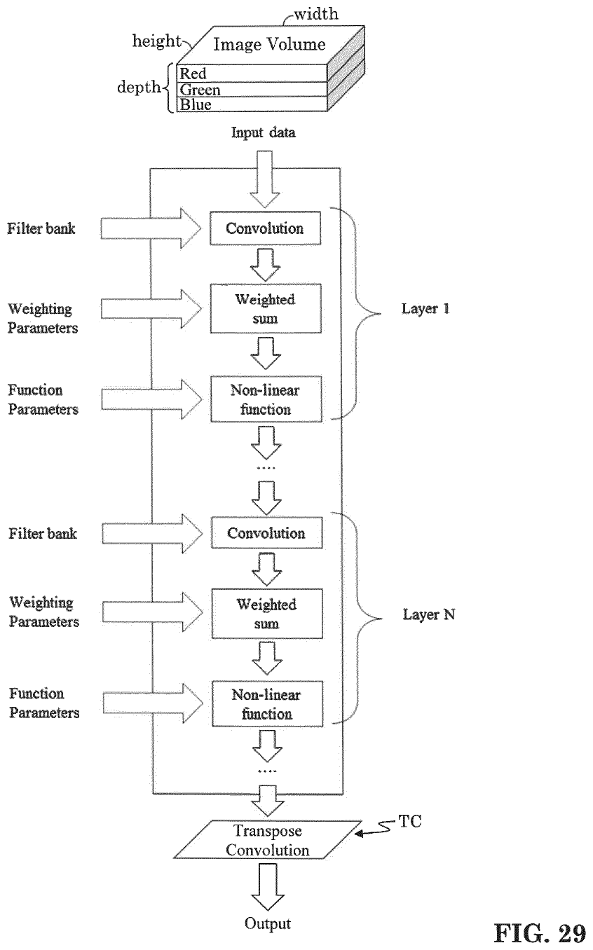

FIG. 29 provides an example convolutional neural network architecture. A convolutional neural network may be defined as a sequence of two or more layers (e.g., Layer 1 to Layer N), where a layer may include a (image) convolution step, a weighted sum (of results) step, and a non-linear function step. The convolution may be performed on its input data by applying a filter (or kernel), e.g. on a moving window across the input data, to produce a feature map. Each layer and component of a layer may have different pre-determined filters (from a filter bank), weights (or weighting parameters), and/or function parameters. In the present example, the input data is an image, which may be raw pixel values of the image, of a given pixel height and width. In the present example, the input image is illustrated as having a depth of three color channels RGB (Red, Green, and Blue). Optionally, the input image may undergo various preprocessing, and the preprocessing results may be input in place of, or in addition to, the raw input image. Some examples of image preprocessing may include: retina blood vessel map segmentation, color space conversion, adaptive histogram equalization, connected components generation, etc. Within a layer, a dot product may be computed between the given weights and a small region they are connected to in the input volume. Many ways of configuring a CNN are known in the art, but as an example, a layer may be configured to apply an elementwise activation function, such as max (0,x) thresholding at zero. A pooling function may be performed (e.g., along the x-y directions) to down-sample a volume. A fully-connected layer may be used to determine the classification output and produce a one-dimensional output vector, which has been found useful for image recognition and classification. However, for image segmentation, the CNN would need to classify each pixel. Since each CNN layers tends to reduce the resolution of the input image, another stage is needed to up-sample the image back to its original resolution. This may be achieved by application of a transpose convolution (or deconvolution) stage TC, which typically does not use any predefine interpolation method, and instead has learnable parameters.

Convolutional Neural Networks have been successfully applied to many computer vision problems. As explained above, training a CNN generally requires a large training dataset. The U-Net architecture is based on CNNs and can generally be trained on a smaller training dataset than conventional CNNs.

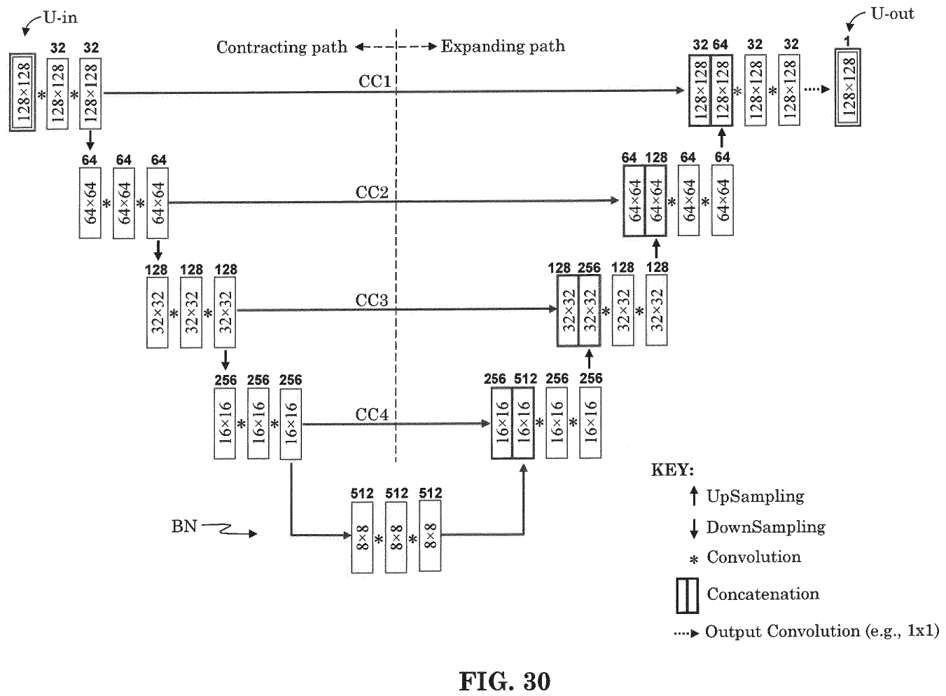

FIG. 30 illustrates an example U-Net architecture. The present exemplary U-Net includes an input module (or input layer or stage) that receives an input U-in (e.g., input image or image patch) of any given size. For illustration purposes, the image size at any stage, or layer, is indicated within a box that represents the image, e.g., the input module encloses number “128×128” to indicate that input image U-in is comprised of 128 by 128 pixels. The input image may be a fundus image, an OCT/OCTA en face, B-scan image, etc. It is to be understood, however, that the input may be of any size or dimension. For example, the input image may be an RGB color image, monochrome image, volume image, etc. The input image undergoes a series of processing layers, each of which is illustrated with exemplary sizes, but these sizes are illustration purposes only and would depend, for example, upon the size of the image, convolution filter, and/or pooling stages. The present architecture consists of a contracting path (herein illustratively comprised of four encoding modules) followed by an expanding path (herein illustratively comprised of four decoding modules), and copy-and-crop links (e.g., CC1 to CC4) between corresponding modules/stages that copy the output of one encoding module in the contracting path and concatenates it to (e.g., appends it to the back of) the up-converted input of a correspond decoding module in the expanding path. This results in a characteristic U-shape, from which the architecture draws its name. Optionally, such as for computational considerations, a “bottleneck” module/stage (BN) may be positioned between the contracting path and the expanding path. The bottleneck BN may consist of two convolutional layers (with batch normalization and optional dropout).

The contracting path is similar to an encoder, and generally captures context (or feature) information by the use of feature maps. In the present example, each encoding module in the contracting path may include two or more convolutional layers, illustratively indicated by an asterisk symbol “*”, and which may be followed by a max pooling layer (e.g., DownSampling layer). For example, input image U-in is illustratively shown to undergo two convolution layers, each with 32 feature maps. As it would be understood, each convolution kernel produces a feature map (e.g., the output from a convolution operation with a given kernel is an image typically termed a “feature map”). For example, input U-in undergoes a first convolution that applies 32 convolution kernels (not shown) to produce an output consisting of 32 respective feature maps. However, as it is known in the art, the number of feature maps produced by a convolution operation may be adjusted (up or down). For example, the number of feature maps may be reduced by averaging groups of feature maps, dropping some feature maps, or other known method of feature map reduction. In the present example, this first convolution is followed by a second convolution whose output is limited to 32 feature maps. Another way to envision feature maps may be to think of the output of a convolution layer as a 3D image whose 2D dimension is given by the listed X-Y planar pixel dimension (e.g., 128×128 pixels), and whose depth is given by the number of feature maps (e.g., 32 planar images deep). Following this analogy, the output of the second convolution (e.g., the output of the first encoding module in the contracting path) may be described as a 128×128×32 image. The output from the second convolution then undergoes a pooling operation, which reduces the 2D dimension of each feature map (e.g., the X and Y dimensions may each be reduced by half). The pooling operation may be embodied within the DownSampling operation, as indicated by a downward arrow. Several pooling methods, such as max pooling, are known in the art and the specific pooling method is not critical to the present invention. The number of feature maps may double at each pooling, starting with 32 feature maps in the first encoding module (or block), 64 in the second encoding module, and so on. The contracting path thus forms a convolutional network consisting of multiple encoding modules (or stages or blocks). As is typical of convolutional networks, each encoding module may provide at least one convolution stage followed by an activation function (e.g., a rectified linear unit (ReLU) or sigmoid layer), not shown, and a max pooling operation. Generally, an activation function introduces non-linearity into a layer (e.g., to help avoid overfitting issues), receives the results of a layer, and determines whether to “activate” the output (e.g., determines whether the value of a given node meets predefined criteria to have an output forwarded to a next layer/node). In summary, the contracting path generally reduces spatial information while increasing feature information.

The expanding path is similar to a decoder, and among other things, may provide localization and spatial information for the results of the contracting path, despite the down sampling and any max-pooling performed in the contracting stage. The expanding path includes multiple decoding modules, where each decoding module concatenates its current up-converted input with the output of a corresponding encoding module. In this manner, feature and spatial information are combined in the expanding path through a sequence of up-convolutions (e.g., UpSampling or transpose convolutions or deconvolutions) and concatenations with high-resolution features from the contracting path (e.g., via CC1 to CC4). Thus, the output of a deconvolution layer is concatenated with the corresponding (optionally cropped) feature map from the contracting path, followed by two convolutional layers and activation function (with optional batch normalization).

The output from the last expanding module in the expanding path may be fed to another processing/training block or layer, such as a classifier block, that may be trained along with the U-Net architecture. Alternatively, or in addition, the output of the last upsampling block (at the end of the expanding path) may be submitted to another convolution (e.g., an output convolution) operation, as indicated by a dotted arrow, before producing its output U-out. The kernel size of output convolution may be selected to reduce the dimensions of the last upsampling block to a desired size. For example, the neural network may have multiple features per pixels right before reaching the output convolution, which may provide a 1×1 convolution operation to combine these multiple features into a single output value per pixel, on a pixel-by-pixel level.

### Computing Device/System

FIG. 31 illustrates an example computer system (or computing device or computer device). In some embodiments, one or more computer systems may provide the functionality described or illustrated herein and/or perform one or more steps of one or more methods described or illustrated herein. The computer system may take any suitable physical form. For example, the computer system may be an embedded computer system, a system-on-chip (SOC), a single-board computer system (SBC) (such as, for example, a computer-on-module (COM) or system-on-module (SOM)), a desktop computer system, a laptop or notebook computer system, a mesh of computer systems, a mobile telephone, a personal digital assistant (PDA), a server, a tablet computer system, an augmented/virtual reality device, or a combination of two or more of these. Where appropriate, the computer system may reside in a cloud, which may include one or more cloud components in one or more networks.

In some embodiments, the computer system may include a processor Cpnt1, memory Cpnt2, storage Cpnt3, an input/output (I/O) interface Cpnt4, a communication interface Cpnt5, and a bus Cpnt6. The computer system may optionally also include a display Cpnt7, such as a computer monitor or screen.

Processor Cpnt1 includes hardware for executing instructions, such as those making up a computer program. For example, processor Cpnt1 may be a central processing unit (CPU) or a general-purpose computing on graphics processing unit (GPGPU). Processor Cpnt1 may retrieve (or fetch) the instructions from an internal register, an internal cache, memory Cpnt2, or storage Cpnt3, decode and execute the instructions, and write one or more results to an internal register, an internal cache, memory Cpnt2, or storage Cpnt3. In particular embodiments, processor Cpnt1 may include one or more internal caches for data, instructions, or addresses. Processor Cpnt1 may include one or more instruction caches, one or more data caches, such as to hold data tables. Instructions in the instruction caches may be copies of instructions in memory Cpnt2 or storage Cpnt3, and the instruction caches may speed up retrieval of those instructions by processor Cpnt1. Processor Cpnt1 may include any suitable number of internal registers, and may include one or more arithmetic logic units (ALUs). Processor Cpnt1 may be a multi-core processor; or include one or more processors Cpnt1. Although this disclosure describes and illustrates a particular processor, this disclosure contemplates any suitable processor.

Memory Cpnt2 may include main memory for storing instructions for processor Cpnt1 to execute or to hold interim data during processing. For example, the computer system may load instructions or data (e.g., data tables) from storage Cpnt3 or from another source (such as another computer system) to memory Cpnt2. Processor Cpnt1 may load the instructions and data from memory Cpnt2 to one or more internal register or internal cache. To execute the instructions, processor Cpnt1 may retrieve and decode the instructions from the internal register or internal cache. During or after execution of the instructions, processor Cpnt1 may write one or more results (which may be intermediate or final results) to the internal register, internal cache, memory Cpnt2 or storage Cpnt3. Bus Cpnt6 may include one or more memory buses (which may each include an address bus and a data bus) and may couple processor Cpnt1 to memory Cpnt2 and/or storage Cpnt3. Optionally, one or more memory management unit (MMU) facilitate data transfers between processor Cpnt1 and memory Cpnt2. Memory Cpnt2 (which may be fast, volatile memory) may include random access memory (RAM), such as dynamic RAM (DRAM) or static RAM (SRAM). Storage Cpnt3 may include long-term or mass storage for data or instructions. Storage Cpnt3 may be internal or external to the computer system, and include one or more of a disk drive (e.g., hard-disk drive, HDD, or solid-state drive, SSD), flash memory, ROM, EPROM, optical disc, magneto-optical disc, magnetic tape, Universal Serial Bus (USB)-accessible drive, or other type of non-volatile memory.

I/O interface Cpnt4 may be software, hardware, or a combination of both, and include one or more interfaces (e.g., serial or parallel communication ports) for communication with I/O devices, which may enable communication with a person (e.g., user). For example, I/O devices may include a keyboard, keypad, microphone, monitor, mouse, printer, scanner, speaker, still camera, stylus, tablet, touch screen, trackball, video camera, another suitable I/O device, or a combination of two or more of these.

Communication interface Cpnt5 may provide network interfaces for communication with other systems or networks. Communication interface Cpnt5 may include a Bluetooth interface or other type of packet-based communication. For example, communication interface Cpnt5 may include a network interface controller (NIC) and/or a wireless NIC or a wireless adapter for communicating with a wireless network. Communication interface Cpnt5 may provide communication with a WI-FI network, an ad hoc network, a personal area network (PAN), a wireless PAN (e.g., a Bluetooth WPAN), a local area network (LAN), a wide area network (WAN), a metropolitan area network (MAN), a cellular telephone network (such as, for example, a Global System for Mobile Communications (GSM) network), the Internet, or a combination of two or more of these.

Bus Cpnt6 may provide a communication link between the above-mentioned components of the computing system. For example, bus Cpnt6 may include an Accelerated Graphics Port (AGP) or other graphics bus, an Enhanced Industry Standard Architecture (EISA) bus, a front-side bus (FSB), a HyperTransport (HT) interconnect, an Industry Standard Architecture (ISA) bus, an InfiniBand bus, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or other suitable bus or a combination of two or more of these.

Although this disclosure describes and illustrates a particular computer system having a particular number of particular components in a particular arrangement, this disclosure contemplates any suitable computer system having any suitable number of any suitable components in any suitable arrangement.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate. While the invention has been described in conjunction with several specific embodiments, it is evident to those skilled in the art that many further alternatives, modifications, and variations will be apparent in light of the foregoing description. Thus, the invention described herein is intended to embrace all such alternatives, modifications, applications and variations as may fall within the spirit and scope of the appended claims.

1. An eye tracking method, comprising:
capturing multiple images of the eye, including a reference image and one or more live images;
defining a reference-anchor point in the reference image;
defining one or more auxiliary points in the reference image;
within a select live image:
a) identifying an initial matching point that matches the reference-anchor point;
b) searching for a match of a select auxiliary point within a region based on the location of the select auxiliary point relative to the reference anchor point; and
correcting for a tracking error between the reference image and the select live image based on their matched points.

2. The method of claim 1, wherein:
a plurality of said auxiliary points are defined in the reference image;
the searching for a match of a select auxiliary point is part of a search for a match of auxiliary points in the select live image;
in response to the number of matched auxiliary points in the select live image not being greater than a predefined minimum, the select live image is not corrected for tracking error.

3. The method of claim 2, the predefined minimum is greater than half the plurality of said auxiliary points.

4. The method of claim 1, wherein the reference image and live images are infrared images.

5. The method of claim 1, wherein the captured multiple images are of the retina of the eye.

6. The method of claim 1, further including:
identifying a prominent physical feature in the reference image;
wherein the reference-anchor point is defined based on the prominent physical feature.

7. The method of claim 6, wherein:
the reference-anchor point is part of a reference-anchor template comprised of a plurality of identifiers that together define the prominent physical feature; and
the identifying of the initial matching point is part of identifying an initial matching template that matches the reference-anchor template.

8. The method of claim 7, wherein:
the one or more auxiliary points is part of a respective one or more auxiliary template comprised of a plurality of identifiers that together define a respective auxiliary physical feature in the reference image; and
the searching for a match of the select auxiliary point is part of searching for a match of a corresponding select auxiliary template within a region based on an offset location of the select auxiliary template relative to the reference anchor template.

9. The method of claim 8, wherein the prominent physical feature in the reference image and in the one or more live image is identified by use of a neural network.

10. The method of claim 9, wherein the prominent physical feature is a predefined retinal structure.

11. The method of claim 10, wherein the prominent physical feature is the optic disc, a lesion, or a specific blood vessel pattern.

12. The method of claim 6, wherein the prominent physical feature is the optic nerve head, pupil, iris boundary or center of the eye.

13. The method of claim 1, further including:
identifying a plurality of candidate anchor points in the reference image;
searching for a match of the plurality of candidate anchor points in the select live image;
designating as said reference-anchor point, the candidate anchor point best matched in the select live image.

14. The method of claim 13, wherein the best matched candidate anchor point is the candidate whose match in the select live image has the highest confidence.

15. The method of claim 13, wherein the best matched candidate anchor point is the candidate whose match is found most quickly.

16. The method of claim 1, further including:
searching for matches of the plurality of candidate anchor points in a plurality of said live images;
designating as said reference-anchor point, the candidate anchor point best matched in the plurality of select live images.

17. The method of claim 16, wherein the best matched candidate anchor point is the candidate anchor point for which a match is most often found in a series of consecutive select live images.

18. The method of claim 17, wherein the series is a predefined number of live images.

19. The method of claim 1, wherein the candidate anchor points are identified based on their prominence within the reference image.

20. The method of claim 1, wherein candidate anchor points not designated as said reference-anchor point are designated auxiliary points.

21. The method of claim 1, further including:
defining a plurality of said reference-anchor points in the reference image;
within the select live image:
a) identifying a plurality of initial matching points that match the plurality of reference-anchor points, and transforming the live image to the reference image as a coarse registration based on the identified plurality of reference-anchor points;
b) searching for a match of a select auxiliary point within a region based on the location of the select auxiliary point relative to the plurality of said reference-anchor points.

22. The method of claim 1, further including:
using an OCT system to define an OCT acquisition field-of-view (FOV) on the eye; wherein:
the reference-anchor point is defined within a tracking FOV moveable within the reference image;
the tracking FOV is moved about the reference image to a position determined to be optimal for image tracking while also at least partially overlapping the OCT FOV.

23. The method of claim 22, wherein the optimal position is based on tracking algorithm outputs including one or more of a tracking error, landmark distribution, and number of landmarks.

24. An eye tracking method, comprising:
capturing multiple images of the retina of an eye, including a reference image and one or more live images;
identifying a prominent physical feature in the reference image;
defining a reference-anchor template based on the prominent physical feature;
defining one or more auxiliary templates based on other physical features in the reference image;
storing the locations of the auxiliary templates relative to the reference-anchor template; within each live image:
a) identifying an initial matching region that matches the reference-anchor template, the initial matching region defining a corresponding live-anchor template whose position is matched to the position of the reference-anchor template;
b) searching for a match of the one or more auxiliary templates, each found match defining another corresponding template in the live image, wherein the search for each auxiliary template is limited to a bound region whose location relative to the live-anchor template is based on the stored location of the auxiliary template relative to the reference-anchor template; and
correcting for a tracking error between the reference image and a select live image based on two or more corresponding templates of the reference image and the select live image.

25. The method of claim 24, wherein the prominent physical feature in the reference image and in each live image is identified by use of a neural network.
